(19) United States
(12) Patent Application Publication
(10) Pub. No.: US 2011/0257245 A1
(43) Pub. Date: Oct. 20, 2011

(54) METHODS OF IDENTIFYING REGULATORS OF CELLULAR PROTEOSTASIS

(75) Inventor: Ron R. Kopito, Stanford, CA (US)

(73) Assignee: The Board of Trustees of the Leland Stanford Junior University, Palo Alto, CA (US)

(21) Appl. No.: 13/090,678

(22) Filed: Apr. 20, 2011

Related U.S. Application Data
(60) Provisional application No. 61/326,224, filed on Apr. 20, 2010.

(51) Int. Cl. A61K 31/7088; C12Q 1/02

(52) U.S. Cl. 514/44 A; 435/29; 514/44 R

(57) ABSTRACT

Embodiments of the present invention provide methods for identifying and utilizing regulators of cellular proteostasis as new therapeutic targets against protein misfolding diseases.

### RELATED APPLICATION

This application claims priority and other benefits from U.S. Provisional Patent Application Ser. No. 61/326,224, filed Apr. 20, 2010, entitled “Methods to identify regulators of cellular proteostasis”. Its entire content is specifically incorporated herein by reference.

### STATEMENT OF GOVERNMENTAL SUPPORT

This invention was made with government support under NS042842 and GM074874 awarded by the National Institutes of Health. The government has certain rights in this invention.

### BACKGROUND

In healthy cells, the ubiquitin-proteasome system (UPS) maintains the well-being of the cell by degrading and clearing improperly folded or otherwise damaged proteins through a large protein complex, the proteasome. The role of the UPS also extends to key regulatory proteins that control various cellular processes such as cell signaling, differentiation and apoptosis and the UPS is therefore considered an essential determinant of cellular function and homeostasis. Loss-of-function mutations on genes that were required for maintenance of ubiquitin (Ub) homeostasis led in mice to the development of neurodegenerative disease, suggesting that impaired proteasome function might contributes to neurodegenerative cytopathology (Ryu et al., 2008; Saigoh et al., 1999; Wilson et al., 2002). Since, however, a mechanistic relationship between protein aggregation and disrupted Ubiquitin homeostasis remains unclear, the involvement of an impaired proteasome in the development of neuropathogenesis remains controversial.

For many years, ubiquitin has been recognized to be a prominent and invariant component of neurofibrillary tangles in Alzheimer's disease, and ubiquitin immunoreactivity is a widely used marker for the postmortem diagnosis of the majority of human neurodegenerative diseases (Cole and Timiras, 1987; Kawasaki et al., 1987; Mori et al., 1987; Perry et al., 1987; Lehman, 2009). In the absence of disease, ubiquitin immunoreactivity is diffusely distributed in brain tissue (Perry et al., 1987). In diseased brain and in cell culture models of neurodegenerative diseases, cellular ubiquitin becomes visibly concentrated within intracellular inclusion bodies (IB), and levels of polyubiquitin chain conjugates are markedly elevated (Mayer et al., 1989; Bennett et al., 2005; Bennett et al., 2007).

A better understanding of the relationship between protein aggregation and disruption of the UPS system might help in the development of therapeutics to target misfolded proteins.

### INCORPORATION BY REFERENCE

All publications and patent applications mentioned in this specification are herein incorporated by reference to the same extent as if each individual publication or patent application was specifically and individually indicated to be incorporated by reference.

### DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates that UPS impairment occurs above a critical, polyglutamine length-dependent concentration of mutant huntingtin, in accordance with embodiments of the present invention. Panel A shows results of a flow cytometry analysis of UPS function in HEK cells: 2-color scatter plots of HEK293 cells (upper row) or HEK293 cells stably expressing UbG76V-GFP (bottom row). Cells were analyzed 72 hrs after transient transfection of htt(Q25)-chFP or htt(Q91)-chFP as indicated. Control experiments included cells incubated overnight with 10 μM MG132 or left untreated (Ctrl). In Panel B, UbG76V-GFP accumulated above a critical concentration threshold of mutant huntingtin. Relationships are indicated between UbG76V-GFP fluorescence (vertical axis) and the fluorescence of htt(Q25)-chFP (), htt(Q91)-chFP (▴) or the difference between the Q91 and Q25 curves (▪) derived from flow cytometry as in panel A. a.u.=arbitrary units. Panel C shows the effect of polyglutamine length on dose-response of chFP-CL1 fluorescence to htt-(Qn)-GFP expression, where n=25 (), 48 (▪), 72 (diamonds ♦) and 91 (▴).

FIG. 2 illustrates that cherry fluorescent protein does not alter the aggregation or UPS impairment phenotypes of huntingtin, in accordance with embodiments of the present invention. Panel A. Fluorescence micrographs of HEK293 cells transiently transfected with htt(Q25)-chFP or htt(Q91)-chFP expressing plasmids. Panel B. Effect of htt(Q25)-chFP (open bars) or htt(Q91)-chFP (filled bars) on GFP-CL1 levels, analyzed by fluorescence microscopy (n>50). Cells expressing huntingtin were identified by red (cherry) fluorescence and total GFP fluorescence was quantified by digital image analysis and plotted as a histogram.

FIG. 3 shows an analysis of flow cytometry data, in accordance with embodiments of the present invention. HEK 293 cells (Panel A) or HEK293 cells stably expressing UbG76V-GFP (Panel B) transfected with htt(Q91)-chFP were analyzed by two color flow cytometry. A set of 41 gates with equal width were set in the chFP channel and the mean GFP fluorescence was calculated for each gate. The GFP histograms that were used to calculate the mean GFP fluorescence for each gate are shown in the right panels. The histograms of the total population of cells are shown left and on top of the scatter plots. The GFP levels attained from the HEK293 cells (A) were then used to calculate the spillover from the chFP channel into the GFP channel for each gate, and to correct the mean GFP fluorescence for the corresponding gate of the UbG76V-GFP expressing cells (B). The same procedure was performed to calculate the corrected GFP means of cells transfected with htt(Q25)-chFP. Histograms and scatter plots (FIGS. 1A and 3) show uncompensated data.

FIG. 4 illustrates that all 26S proteasome substrates are stabilized at the same critical concentration threshold of mutant huntingtin expression, in accordance with embodiments of the present invention. Panel A. All cytoplasmic UPS reporters accumulated above the same critical concentration threshold of mutant huntingtin expression, as shown by the effect of htt(Q91)-chFP expression x-axis) on the fluorescence levels of different unstable GFP-degron constructs (y-axis): UFD substrate UbG76V-GFP (▪), Ub-independent substrate cODC-GFP (diamonds ♦), N-end rule substrate Ub-R-GFP (▴), and CL1-dependent substrate, GFP-CL1 (). Panel B. ERAD substrates accumulated above the same critical concentration threshold of mutant huntingtin expression. Relationship between htt(Q91)-chFP expression x-axis) plotted against the fluorescence levels of GFP-tagged ERAD substrates (y-axis) are: GFP-ΔF508 (diamonds ♦) and TCRα-GFP (▴). UbG76V-GFP (▪) was included for comparison.

FIG. 5 illustrates that ubiquitinated polyglutamine aggregates do not choke the 26S proteasome in vitro, in accordance with embodiments of the present invention. Panel A shows in vitro ubiquitination of radiolabeled Sic1 substrate. [35S]PY-Sic1, purified from E. coli was ubiquitinated in vitro as described in Experimental Procedures. Aliquots of the reaction were removed at the indicated times, separated on a 4-20% gradient gel, and visualized by autoradiography. Mobilities of unmodified Sic1 and polyubiquitinated Sic1 are indicated. Panel B shows an kinetic analysis of Sic1-Ubn degradation. [35S]Sic1-Ubn (100 nM) was incubated in the presence of 10 nM 26S proteasomes and degradation kinetics were assessed by SDS-PAGE or release of TCA-soluble 35S radioactivity. Initial rates of substrate degradation were determined from the kinetics of 35S radiolabel release and fitted to the Michaels-Menten equation by least squares analysis assuming a Km of 50 nM. Panel C shows the preparation of ubiquitinated MPC, an N-terminal maltose binding protein (MBP) fused to a fragment of Xenopus cyclin (cyclin N100) and a T7 epitope tag. MPC was purified from E. coli and was ubiquitinated in vitro as described in Experimental Procedures, infra. Aliquots of the reaction were removed at the indicated times, separated on a 4-20% gradient gel, and visualized by immunoblotting with anti-T7 horse radish peroxidase conjugate. Panel D. Ubiquitinated MPC is a competitive inhibitor of Sic1-Ubn degradation. The initial rate of [35S]Sic1-Ubn degradation depends on the concentration of MPC (closed circles) or ubiquitinated MPC (MPC-Ubn) (open circles). Proteasomes (10 nM) were incubated with 100 nM substrate and initial rates were determined by a linear fit to soluble TCA radioactivity. Initial rates are expressed as a percentage of the control reaction without MPC or MPC-Ubn. The data were fit by least-squares analysis as described in Experimental Procedures, infra. Panel E shows the preparation of ubiquitinated huntingtin (htt) fragments. GST-PY-htt(Qn) containing a C-terminal S-tag was purified from E. coli and ubiquitinated in vitro as described in Experimental Procedures, infra. Aliquots of the reaction were removed at the indicated times, treated as indicated with the deubiquitinating enzyme Usp2, separated on a 4-20% gradient gel, and visualized by immunoblotting with anti-T7 horse radish peroxidase conjugate. Panel F. Competitive inhibition of Sic1-Ubn degradation by ubiquitinated huntingtin is independent of polyglutamine length or aggregation state. The initial rate of [35S]Sic1-Ubn degradation depends on the concentration of htt(Q18)-Ubn (top panel) or htt(Q51)-Ubn (bottom panel). Analysis was performed with non-aggregated (uncleaved) htt(Q51)-Ubn (closed circles) or following TEV cleavage and aggregation of the ubiquitinated htt(Q51)-Ubn (open circles) was analyzed. Proteasomes (10 nM) were incubated with 100 nM substrate and initial rates were determined by a linear fit of soluble TCA radioactivity. Initial rates are expressed as a percentage of the control reaction without inhibitor present. The data were fit by least-squares analysis as described in Experimental Procedures, infra. Panel G shows that htt(Q51)-Ubn aggregates are insoluble. htt(Q51)-Ubn was aggregated and filtered through a 0.2 μm cellulose acetate filter as described in Experimental Procedures, infra. Blot was probed with anti-Ub (FK2) monoclonal antibody (top) or S-protein-HRP to detect polyubiquitinated trapped huntingtin aggregates.

FIG. 6 depicts features of fluorescent proteasome reporters, used in embodiments of the present invention. Untransfected HEK293 cells, and cells stably expressing GFP-CL1, UbG76V-GFP, Ub-R-GFP or cODC-GFP were treated with 10 μM MG-132 and at the times indicated, mean GFP fluorescence was analyzed by flow cytometry (Panel A) or by fluorescence microscopy of living cells (Panel B). Panel C shows that cODC-GFP degradation is Ub-independent. ts20 cells harboring a thermosensitive Ub activation enzyme, E1, were stably transfected with the indicated destabilized GFP variant. Cells were incubated overnight at the permissive temperature 32° C. (black bars), at 32° C. in the presence of 10 μM MG-132 (white bars) or at the non-permissive temperature 40° C. (grey bars). Mean GFP fluorescence was analyzed by flow cytometry, and plotted as relative fluorescence change. Panel D: Htt(Q91)-chFP expression does not affect steady-state levels of a stable GFP reporter. HEK293 cells stably expressing UbG76V-GFP or GFP were transiently transfected with httchFP, and analyzed as described in FIG. 1B. The ordinate shows the difference of the means of the compensated GFP fluorescence between htt(Q91)-chFP and htt(Q25)-chFP for a given concentration of htt-chFP. Htt(Q91)-chFP-specific GFP fluorescence is plotted for GFP (♦) and UbG76V-GFP expressing cells (▪).

FIG. 7 illustrates that the stabilization of proteasome reporters by aggregation-prone huntingtin is not due to direct competition for 26S, in accordance with embodiments of the present invention. Panel A. Short-lived proteasome substrates, but not huntingtin, competed for degradation of chFP-CL1 degradation. HEK293 cells stably expressing chFP-CL1 were transfected with GFP-CL1 (▪), cODC-GFP (diamonds ♦), GFP (x), htt(Q25)-GFP () or htt(Q91)-GFP (▴) and analyzed by 2-color flow cytometry. Inset, double logarithmic plot shows that cODC-GFP and GFP-CL1 data fit well with a linear least squares regression (r2>0.98), while htt(Q91)-GFP does not. Panel B. Cytoplasmic NES-GFP-CL1 does not effectively stabilize chFP-CL1. HEK293 cells stably expressing chFP-CL1 and transfected with either GFP-CL1(▪) or NES-GFP-CL1 (□) were analyzed by flow cytometry as in panel A.

FIG. 8 illustrates that HSF1 activation suppresses the effect of mutant huntingtin on stabilization of UbG76VGFP, in accordance with embodiments of the present invention. Panel A shows the effect of thermal stress on htt(Q91)-chFP-specific accumulation of UbG76V-GFP. HEK293 cells stably expressing UbG76V-GFP were transfected with htt(Q91)-chFP or htt(Q25)-chFP and analyzed by flow cytometry after overnight incubation at either 37° C. (Ctrl, ▪) or 40° C. (▴). Panel B. Effect of geldanamycin on htt(Q91)-chFP-specific accumulation of UbG76V-GFP. HEK293 cells stably expressing UbG76V-GFP were transfected with htt(Q91)-chFP or htt(Q25)-chFP and analyzed by flow cytometry after overnight incubation with (▴) or without geldanamycin (500 nM; Ctrl, ▪). Panel C shows the effect of HSF1 overexpression on htt(Q91)-chFP-specific accumulation of UbG76VGFP. HEK293 cells stably expressing UbG76V-GFP were co-transfected with htt(Q91)-chFP or htt(Q25)-chFP together with wildtype (▴) or constitutively active (diamonds ♦) HSF1 and analyzed by flow cytometry.

FIG. 9 illustrates that persistent expression of mutant huntingtin does not activate the heat-shock response, in accordance with embodiments of the present invention. Panel A: Fluorescent heat shock reporter responds to activation of the heat shock response. Normalized GFP fluorescence in HEK293 cells stably expressing Hsp70::GFP reporter following heat shock or treatment with celastrol (1 μM). Cells were incubated overnight in the indicated condition prior to analysis of mean GFP fluorescence by flow cytometry. Panel B: Expression of mutant huntingtin does not induce the heat shock response. HEK293 cells stably expressing UbG76V-GFP (▪) or Hsp70::GFP (diamonds ♦) were transfected with htt(Q91)-chFP or htt(Q25)-chFP and analyzed by flow cytometry. Panel C: Effect of equivalent levels of expression of mutant huntingtin expression on UbG76VGFP accumulation becomes more severe with increasing expression time. HEK293 cells stably expressing UbG76V-GFP were transfected with htt(Q91)-chFP or htt (Q25)-chFP and analyzed by flow cytometry at 48 (), 72 (▪), 96 (diamonds ♦), or 120 (▴) hrs following transfection.

FIG. 10 illustrates the kinetics of radiolabeled Sic1 degradation by 26S proteasomes, in accordance with embodiments of the present invention. Panel A: SDS-PAGE analysis of [35S]PY-Sic1 degradation. Radiolabeled (100 nM) substrate was incubated with or without 10 nM 26S proteasomes for the indicated times in the presence or absence of 10 μM MG132. The reaction products were separated on a 4-20% gradient gel, and visualized by autoradiography. Mobilities of unmodified Sic1 and polyubiquitinated Sic1 are indicated. Note the appearance of de-ubiquitinated substrate when the reaction was performed in the presence of the active site inhibitor MG132. Panel B: Kinetics of [35S]PY-Sic1 degradation. Radiolabeled Sic1-Ubn substrate was incubated with 10 nM 26S proteasomes either before (∘) or after () treatment with the de-ubiquitinating enzyme Usp2, in the absence or presence (□) of MG132. Aliquots of the reaction were removed at the indicated times and analyzed for TCA soluble radioactivity. Percent degradation is expressed as [(measured TCA soluble counts)/(measured water soluble counts)×100] and fitted to the Michaelis Menten equation assuming a Km of 50 nM. The linear initial rate of the degradation curve (box) is expanded in the lower plot.

FIG. 11 illustrates the aggregation of PY-htt(Q51) in vitro, in accordance with embodiments of the present invention. GST-PY-htt(Q51) containing a Cterminal S-tag was purified from E. coli and incubated with or without TEV protease as indicated. Aliquots of the reaction were removed at the indicated times and analyzed on a 4-20% SDS-PAGE gradient gel (top) or 0.2 μm cellulose acetate filter trap (bottom) and visualized by detection with S-protein horse radish peroxidase conjugate.

### DEFINITIONS

The practice of the present invention may employ conventional techniques of chemistry, molecular biology, recombinant DNA, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique', Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable.

The term “proteostasis”, as used herein, refers to a complex network (‘proteostatic systems’) which integrates the biosynthesis, folding and assembly of proteins with the regulatory machinery that ensures fidelity in these processes. Hereby, this complex network encompasses (i) molecular chaperones and enzymes including, but not limited to isomerases, peptidases, glycosylating enzymes and the like, all which covalently modify intracellular proteins, (ii) regulatory factors such as kinases and phosphatases that control protein synthesis, (iii) proteolytic systems including the ubiquitin-proteasome system (UPS) and lysosomal autophagy which degrade improperly folded proteins.

The term “adequate proteostasis capacity”, as used herein, refers to the ability of cells to maintain proteostasis.

The term “impaired proteostasis capacity”, as used herein, refers to the inability of cells to maintain proteostasis.

The term “cytometry”, as used herein, refers to a process in which physical and/or chemical characteristics of single cells, or by extension, of other biological or nonbiological particles in roughly the same size or stage, are measured. In flow cytometry, the measurements are made as the cells or particles pass through the measuring apparatus (“flow cytometer”) in a fluid stream. A cell sorter, or flow sorter, is a flow cytometer that uses electrical and/or mechanical means to divert and collect cells (or other small particles) with measured characteristics that fall within a user-selected range of values.

### DETAILED DESCRIPTION

Embodiments of the present invention provide methods for identifying regulators of cellular proteostasis as new therapeutic targets for treating various protein misfolding diseases.

In one aspect of the present invention, a reporter of ubiquitin-proteasome system (UPS) activity (a ‘UPS reporter’) is used to assess the proteostatic condition and health of a cell by correlating UPS function or dysfunction with the expression of a protein-misfolding disease-related folding-compromised protein such as huntingtin. Through its role of integrating multiple proteostatic subnetworks, the UPS reporter can serve as a marker to capture when cellular proteostasis goes out of balance.

In one aspect of the present invention, a UPS reporter, which can be fluorescent (such as green) or nonfluorescent and containing a mutant form of ubiquitin, is expressed in engineered cells, such as mammalian cells, in combination with an aggregation-prone protein (aggregant) that is characteristic of a protein misfolding disease. In one embodiment of the present invention, the aggregation-prone protein is huntingtin. Fluorescent proteins are chosen such that their fluorescent properties are compatible, but don't overlap with the UPS reporter; color compensation is carried out, whenever appropriate.

In another aspect of the present invention, a modified aggregant protein containing a sub-pathogenic member is coexpressed with a UPS reporter in engineered cells to indicate threshold levels. In one embodiment of the invention, a modified huntingtin protein is used to obtain such threshold levels for UPS reporter expression, whereby an N-terminal fragment of huntingtin containing a sub-pathogenic polyglutamine &#8805 40 glutamine) tract is fused to a fluorescent (red) or nonfluorescent protein, serving as a control to set threshold levels.

In a further aspect of the invention, a population of cells, programmed to express a particular aggregation-prone protein (aggregant), is analyzed in the presence of a potential regulator of proteostasis to differentiate populations of cells that have maintained adequate proteostasis capacity from populations of cells that have impaired proteostasis. A successful regulator of proteostasis would prevent impairment of proteostasis.

### Protein Misfolding Diseases (Proteinopathies or Proteopathies)

Proteostatic (protein homeostatic) systems are believed to have become dysregulated in protein misfolding diseases. Protein misfolding diseases describe conditions where certain proteins in the body have become structurally abnormal through misfolding, i.e. not folding into their regular configuration for being functional, as well as aggregating all of which, as a consequence, disrupt the proper functioning of the concerned cells, tissues and organs or even exert some toxic effects on these cells, tissues and organs. Very often, misfolded proteins exhibit an increased tendency to bind to themselves and, thus, to aggregate (Carrell & Lomas, 1997).

Protein misfolding diseases encompass neurodegenerative disorders and diseases that are associated and characterized by the formation of toxic, intracellular or extracellular protein deposits including, but not limited to, Alzheimer's disease, Parkinson's disease, Huntington's disease, Lewy body dementia, prion diseases, tauopathies, and serpinopathies. Protein misfolding diseases also encompass disorders such as type-2 diabetes, which can involve a mutation of the islet amyloid polypeptide gene with resulting misfolding of the protein, and liver diseases.

Amyloidogenic Diseases (Amyloidoses)

Amyloidogenic diseases including Alzheimer's disease encompass pathological conditions that are characterized by an accumulation of beta-amyloid (Aβ) peptides or related amyloidogenic peptides or proteins as intracellular or extracellular protein deposits. Amyloidogenic diseases are typically localized, but can occur in systemic form as well.

Alzheimer's disease (AD) is a devastating, degenerative disorder of the brain and the leading cause of dementia in the elderly, that phenotypically starts with memory loss and eventually results in complete loss of intellectual and everyday life skills Deposits of various β-amyloid peptides (Aβ) in the form of extracellular plaques and the generation of neurofibrillary tangles in the human brain are the most prevalent histopathological hallmarks of AD.

Certain familial forms of Alzheimer's disease, as well as Down's syndrome, are the result of mutations in beta amyloid precursor protein, resulting in deposition of plaques having fibrils composed mainly of β-amyloid peptide (Aβ); symptoms are then observable in individuals already in their thirties or forties.

Familial British dementia (FBD) or familial Danish dementia (FDD) are characterized by progressive cognitive impairment, spasticity, and cerebellar ataxia with neurofibrillar degeneration and widespread parenchymal and vascular amyloid deposits. These forms of dementia are associated with a mutation in the BRI gene, resulting in the production of an amyloidogenic fragment, amyloid-Bri (ABri) for Familial British dementia and Adan for familial Danish dementia.

Huntington's Disease

Huntington's disease is an autosomal dominant neurodegenerative disorder that results from brain damage caused by aggregats of misfolded huntingtin protein and that affects muscle coordination and cognitive functions, typically from middle age on. A prominent characteristic of Huntington's disease is the presence of protein aggregates, in particular of huntingtin protein aggregates, that are collected in inclusion bodies. As mentioned earlier, such intracellular inclusion bodies also contain components of the ubiquitin-proteasome system.

Parkinson's Disease and Lewy Body Dementia

Lewy body dementia, a synucleinopathy, is closely associated with both Alzheimer's and Parkinson's diseases and is characterized anatomically by the presence of Lewy bodies, which are cytoplasmic inclusions of alpha-synuclein and ubiquitin protein, in neurons.

Parkinson's disease is a degenerative disorder of the central nervous system that primarily impairs motor skills and speech. These symptoms result from decreased stimulation of the motor cortex due to insufficient production of dopamine in dopaminergic neurons of the brain. It is assumed that the presence of Lewy bodies contributes to the gradual death of brain cells and tissue.

Prion Diseases

Prion diseases, also known as transmissible spongiform encephalopathies, are characterized by deposits of abnormally shaped prion proteins in the brain leading to neurodegeneration. Familial forms of prion disease are caused by inherited mutations in the PRNP gene.

Tauopathies

Tauopathies are neurodegenerative disorders that result from the toxic aggregation of tau protein in neurofibrillary tangles in the brain.

Serpinopathies

Serpinopathies are disorders that affect the central nervous system and beyond and that results from pathological polymerization and precipitation of serpin proteins. Well-characterized serpinopathies include alpha 1-antitrypsin deficiency, which may cause familial emphysema and sometimes liver cirrhosis, certain familial forms of thrombosis related to antithrombin deficiency and familial encephalopathy with neuroserpin inclusion bodies.

Protein Homeostasis

Protein homeostasis or proteostasis refers to the regulatory network that coordinates the expression of molecular chaperones, folding enzymes and degradative machinery with the amount and type of proteins present in a cell or cellular compartment under changing metabolic, environmental and developmentally programmed conditions (Balch et al., 2008). A key function of the proteostasis network is to ensure that expression of molecular chaperones is commensurate with the degree of “client” demand.

It is of great importance for the health of a cell to regulate the individual entities of its proteome as well as to control the structural fidelity of each of its members. The regulation and maintenance of protein homeostasis plays an essential role in preventing protein aggregation.

### Protein Aggregation and Impairment of Ups Activity

One approach to assess the impact of pathogenic aggregation-prone proteins, that are the cause of proteinopathies, on the ubiquitin-proteasome system (UPS) has been to use synthetic “reporters” consisting of green fluorescent protein (GFP) fused to destabilizing degrons that convert GFP from a very long-lived protein to a shortlived, ubiquitin-dependent proteasome substrate (Bence et al., 2001; Dantuma et al., 2000). In cells expressing these reporters, steady-state levels of GFP fluorescence provide—assuming constant synthesis rates—a readout of UPS functional capacity. Multiple studies have reported that the levels of these reporters, when expressed in mammalian cell culture, increase significantly upon co-expression of aggregation-prone mutant, but not wild-type variants of neurodegenerative disease-linked proteins including huntingtin (Bence et al., 2001), ataxin-1 (Bennett et al., 2005), androgen receptor (Mandrusiak et al., 2003), PrP (Deriziotis and Tabrizi, 2008), α-synuclein (Petrucelli et al., 2002), UBB+1 (van Tijn et al., 2007) and rhodopsin (Illing et al., 2002). While these findings support the conclusion that protein aggregation and UPS impairment are intimately related, they provide scant insight into the possible mechanisms underlying this relationship.

The inventor of the present invention and his colleagues have previously used quantitative microscopy to show that the unstable UPS reporter GFP-CL1 accumulates in cells expressing huntingtin fragments bearing pathogenic (Q103) but not wild-type (Q25) polyglutamine repeats (Bence et al., 2001). In those studies, the amount of GFP-CL1 fluorescence was found to correlate with the diameter of huntingtin-positive IB, suggesting a relationship between the amount of mutant huntingtin aggregation (or expression) and the extent of UPS impairment (Bence et al., 2001). Subsequently, it was demonstrated that GFP-CL1 levels were elevated—albeit to a lesser extent—in cells expressing mutant huntingtin but lacking microscopically visible IB, suggesting that IB formation is not essential for UPS impairment (Bennett et al., 2005). Upon challenge with chemical proteasome inhibitors (Ryu et al., 2006) or overexpression of aggregation-prone proteins fragments such as mutant huntingtin bearing a pathogenically expanded polyglutamine tract, a substantially increased fraction of cellular Ub was present in polyubiquitin conjugates that was observable as a high molecular weight “smear” on Ub immunoblots (Bennett et al., 2007) or by polyubiquitin-selective ubiquitin association (hP2UBA) affinity capture coupled to analysis of linkage-specific Ub-Ub isopeptides by mass spectrometry (MS) analysis (Bennett et al., 2007). Quantitative mass spectrometry has been used to document elevated polyubiquitin content in brains from R6/2 Huntington's disease (HD) model mice, and in postmortem brain from HD patients (Bennett et al., 2007). This increase can be detected in R6/2 mice at 6 wks of age, corresponding roughly to the time of neuropathological onset in this HD model. Focal accumulation of polyubiquitin immunoreactivity in IB, elevated levels of endogenous polyubiquitin chains and stabilization of fluorescent, short-lived UPS “reporters” in cell and animal models of HD and other neurodegenerative disorders all indicate that protein aggregation is associated with severe UPS impairment.

The simplest mechanism by which protein aggregation could result in accumulation of polyubiquitinated proteins would be if aggregates directly inhibited or “choked” 26S. Although previous reports concluded that proteasomes are unable to cleave within polyglutamine tracts, raising the possibility that indigestible aggregates may choke proteasomes by direct interaction (Holmberg et al., 2004; Venkatraman et al., 2004), more recent studies have failed to confirm this conclusion (Pratt and Rechsteiner, 2008). Moreover, pure polyglutamine peptides—whether soluble or aggregated—do not influence either 20S or 26S activity in vitro (Bennett et al., 2005). It is possible, however, that polyubiquitinated protein aggregates could choke proteasomes by engaging in a “tug-of-war” between polyubiquitin-mediated substrate binding and futile attempts by the 19S AAA ATPases to unfold the highly aggregated polyglutamine tracts.

### Drug Screening Methods to Identify Regulators of Proteostasis

Drug screening methods generally involve conducting various types of assays to identify agents that alter the proteostatic capacity of cells. A library of compounds consisting of small interfering ribonucleic acids (siRNAs), complementary deoxyribonucleic acids (cDNAs) or small molecules can be screened for agents with a potential utility in normalizing the proteostatic capacity of a cell, whereby normalizing the proteostatic capacity means reinstating regular cellular proteostasis. The library of compounds can be commercially available, can be proprietary, or can be custom synthesized. siRNAs are typically around 15-30 nucleotides in length and can contain areas of hybridizability with genes that encode for aggregation-prone proteins, such as huntingtin.

Determining the in vivo efficacy of candidate compounds is also contemplated. Candidate compounds may be administered to an animal in a model for a neurodegenerative disease.

### A Method to Identify Regulators of Cellular Proteostasis

In various embodiments of the present invention, a reporter of ubiquitin-proteasome system (UPS) activity (a ‘UPS reporter’) is used to assess the proteostatic condition and health of a cell by correlating UPS function or dysfunction with the expression of a protein-misfolding disease-related folding-compromised protein. Through its role of integrating multiple proteostatic subnetworks, the UPS reporter can serve as a marker to capture when cellular proteostasis is or becomes imbalanced.

Regulators of cellular protein homeostasis (proteostasis) act to maintain the health of a cell by maintaining protein homeostasis or by restoring, to some degree or to a full degree, protein homeostasis. Regulators of cellular protein homeostasis can be identified in cells, such as mammalian cells, that simultaneously express (co-express) a ubiquitin-proteasome system (UPS) reporter and an aggregation-prone protein. If a candidate agent is capable of regulating cellular protein homeostasis, UPS reporter expression will not be altered to a statistically significant degree, if such a molecule is contacted with a cell that coexpresses a UPS reporter and an aggregation-prone protein such as huntingtin, in comparison to controls. If a test molecule is not capable of regulating cellular protein homeostasis, UPS reporter expression will be altered to a statistically significant degree, if such a molecule is contacted with a cell that coexpresses a UPS reporter and an aggregation-prone protein such as huntingtin, in comparison to controls.

### A Method of Reducing Aggregation of an Aggregation-Prone Protein, The Method Comprising Administering to a Mammal a Regulator of Cellular Protein Homeostasis Identified in Accordance to Claim 1.

Regulators of cellular proteostasis hold the promise to exert a beneficial effect in reducing the impact of protein misfolding diseases when administered to a mammal who carries a genetically based risk of developing a protein misfolding disease, such as Huntington's Disease, or who already suffers from a protein misfolding disease.

In the following, experimental procedures and examples will be described to illustrate parts of the invention.

### EXPERIMENTAL PROCEDURES

The following methods and materials were used in the examples that are described further below.

Plasmids

The plasmids expressing GFP-CL1 and Huntingtin exon 1-GFP were described previously (Bence et al., 2001). Plasmids expressing Ub-R-GFP and UbG76V-GFP were kind gifts of Nico Dantuma (Karolinska Institute, Stockholm, Sweden). The plasmids expressing HSF(wt) and the dominant positive mutant HSF(d203-315) were kind gifts of Richard Voellmy (University of Miami, Miami, Fla.). The cODC-GFP expressing plasmid was created by amplification of the C-terminal 37 amino acids of ODC by PCR and cloned into pEGFP1 (Clontech, Mountain View, Calif.) using the HindIII and BamHI sites. Plasmids expressing HttQ25-chFP and HttQ91-chFP were created by inserting chFP (a kind gift of Roger Tsien, University of California at San Diego, La Jolla, Calif.) into the BamHI-site of the Htt exon 1 plasmids described previously (Bence et al., 2001). Hsp70 GFP plasmid was created by inserting the hsp70 promotor from the Hsp70.1-pr-Luciferase plasmid (a kind gift from Richard Morimoto, Northwestern University, Evanston, Ill.) into a modified promotorless pEGFP-C3 (Clontech, Mountain View, Calif.). The chFP-CL1 plasmid was created by insertion of chFP and the CL-1 sequence into pcDNA3.1 (Invitrogen, Carlsbad, Calif.). pET3a-Ub and pET15b-Ubc4, were kindly provided by the late Cecile Pickart (Johns Hopkins University, Baltimore, Md.) and Daniela Rotin (The Hospital for Sick Children, Toronto, Canada), respectively. pHUE and pHUsp2-cc were gifts from Rohan Baker (The Australia National University, Can berra, Australia). Plasmids for GST-Rsp5 and GST ΔC2Rsp5 were obtained from John Huibregtse (University of Texas, Austin, Tex.). The ΔC2Rsp5 coding sequence was PCR amplified and cloned into a pET28a vector (Novagen). The plasmid for expressing His6-PYSic1 was obtained from Raymond Deshaies (Howard Hughes Medical Institute, CalTech, Pasadena, Calif.). The PYSic1 coding sequence was PCR amplified and cloned into the pHUE vector by standard methods as described (Catanzariti et al., 2004). A plasmid containing the cyclinB N100 fragment {Chen, 2001 #4200) was obtained from Guowei Fang (Stanford University). The cyclinB N100 coding sequence was PCR amplified and cloned into the pMAL-c2X plasmid (New England Biolabs). Site-directed mutagenesis was used to introduce a Pro-Pro-Pro-Tyr sequence (PY motif) infront of the cyclinN100 sequence and a T7-tag at the end of cyclinN100 to create pMAL-PY-cyclinN100-T7 (MPC). Plasmids expressing the original GST-huntingtin fusion constructs containing either Q18 or Q51 were obtained from Erich Wanker (Max Delbrueck Center, Berlin, Germany). A TEV protease site was introduced between the GST and the huntingtin Q51 fragment (GST-HttQn-ΔS). Plasmids for GST-fused huntingtin Q18 or Q51 containing a TEV protease site between GST and the huntingtin exon1 fragment and a C-terminal S-tag were previously described (Bennett et al., 2005). Site-directed mutagenesis was used to insert a PY motif immediately after the TEV protease site to create GST-PYHtt (Q18)-S and GST-PY-Htt(Q51)-S constructs.

Cell Lines

The GFP-CL1 and CFTRΔF508-GFP cell lines were described earlier (Bence et al., 2001; Johnston et al., 1998). Stable HEK293 cell lines expressing the constructs described herein were created by transfection, selection with G-418 and cloned by limiting dilution. The temperature sensitive ts20 Balb/C 3T3 clone A31 fibroblast cell line (Kulka et al., 1988) was a kind gift of DT Madden (The Buck Institute for Age research, Novato, Calif.). All cells were grown in DMEM with 10% animal serum complex, L-glutamine and antibiotics.

Flow Cytometry

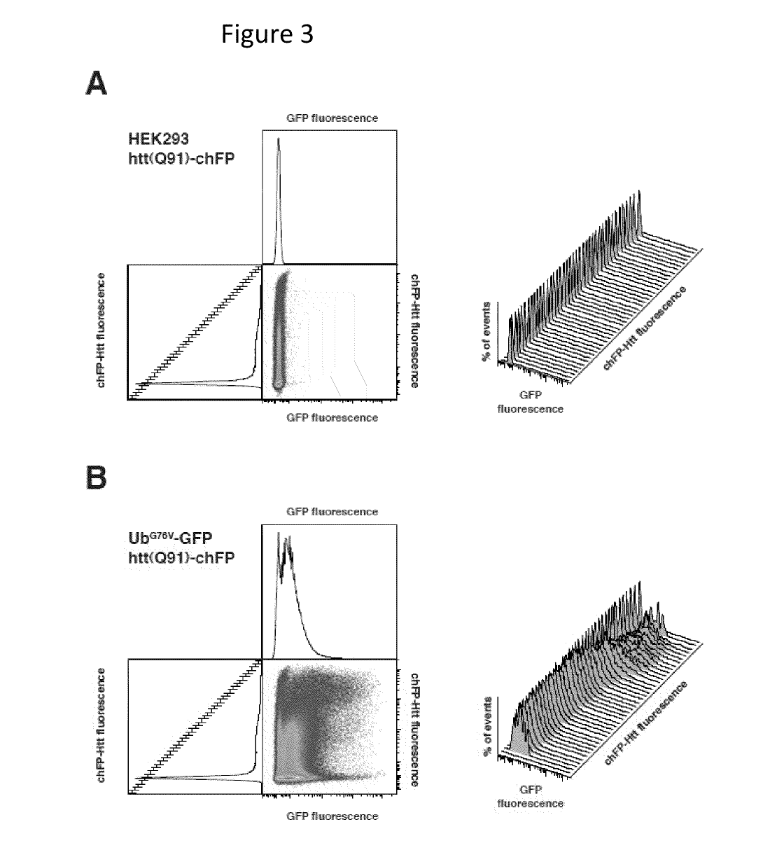

Unless indicated, cells were harvested 72 hrs after transfection and analyzed with a Becton-Dickinson LSRII flow cytometer with a 488 nm and a 535 nm Laser (BD, Franklin Lakes, N.J.). To ensure a sufficient number of cells with elevated levels of the transfected protein, over 200,000 cells were analyzed per condition in a typical experiment. To plot the level of the reporter-protein versus the level of the transfected protein (described in FIG. 3), a set of 41 of gates of equal width (on a logarithmic scale) was set up in the channel for the transfected protein. The mean compensated fluorescence of the reporter protein in each of these gates was calculated and plotted on the ordinate with the gate number (corresponding to the log of fluorescence intensity of the transfected protein) plotted on the abscissa. Each construct and condition in singly transfected HEK293 cells was used as a single color control to compensate the spillover between chFP and GFP individually for each gate. Gates with less than 100 events were not included in the analysis. Raw flow cytometry data were analyzed using FlowJo (version 8.8.6, Tree Star) software.

Protein Purification

Recombinant ubiquitin, recombinant Ubc4, GST-Rsp5, and Usp2 cc were purified as previously described (Catanzariti et al., 2004; Thrower et al., 2000). Recombinant human E1 enzyme was purchased from Boston Biochem. The ΔC2-Rsp5 protein was expressed in E. coli and purified by using Talon metal affinity chromatography (Clontech, Mountain View, Calif.). Briefly, cell pellets were resuspended in 40 vol of 50 mM Tris-HCl pH 7.6, 300 mM NaCl, 5 mM β-mercaptoethanol, 1 mM PMSF and lysed by sonication. The lysate was clarified at 10,000×g for 30 min before being loaded onto a gravity column packed with 3 mL of Talon beads. The column was washed with 20 column volumes of resuspension buffer without β-mercaptoethanol, and eluted with 3 column volumes of 50 mM Tris, pH 7.6, 300 mM NaCl, 200 mM imidazole, and 10% glycerol. The eluted protein was dialyzed into 50 mM Tris-HCl pH 7.6, 150 mM NaCl, 1 mM DTT, and 10% glycerol. GST-HttQ51ΔS, GST-PY-HttQ18-S, and GST-PY-HttQ51-S were affinity purified with Glutathione Sepharose 4b resin (GE Healthcare Life Sciences). PYSic1 was expressed as an N-terminal His6-tagged ubiquitin fusion protein and metabolically labeled in E. coli grown in minimal media supplemented with [35S]-methionine as previously described (Zhang et al., 2003). The radiolabeled, ubiquitin-fusion protein was bound to Talon metal affinity resin, cleaved with Usp2 cc, and eluted as previously described (Catanzariti et al., 2004). MPC was affinity purified with amylose resin according to the manufacturer (New England Biolabs). 26S proteasomes were purified from Sprague Dawley rats as previously described (Zhang et al., 2003).

Substrate Ubiquitination

0.05-0.2 mg/mL of [35S]-PYSic1 and MPC were ubiquitinated for 16 hrs in the presence of 50 nM E1 (Boston Biochem), 2.4 μM E2 (His6-Ubc4), 2 μM E3 (GST-Rsp5), 300 μM ubiquitin, and 1×ATP buffer (40 mM Tris-HCl pH 7.2, 2 mM ATP, 5 mM MgCl2, 1 mM DTT). The reaction was then mixed with 0.5 vol of glutathione sepharose beads for 3 hr at RT to remove contaminating autoubiquitinated GST-Rsp5. Similarly, 0.05-0.2 mg/mL GST-PYHttQn-S were ubiquitinated except that 2 μM E3 (ΔC2Rsp5) was used and 1 vol of Talon metal affinity resin was used to remove any autoubiquitinated E3 enzyme. [35S]-PYSic1 ubiquitination ([35S]Sic1-Ubn) was monitored by SDS-PAGE and autoradiography. The ubiquitination of MPC (MPC-Ubn) was monitored by SDS-PAGE and detected with T7-HRP conjugate (Pierce) followed by chemiluminescence. The ubiquitination of GST-PY-HttQn-S was monitored by separation on SDS-PAGE and detection with S-HRP conjugate (Novagen) followed by chemiluminescence.

In Vitro Sic1 Degradation Assay

The substrate degradation reaction was carried out at room temperature in 50-100 μL total volume containing 10 nM 26S, 1×ATP buffer, and 100 nM [35S]Sic1-Ubn. 5 μL reaction aliquots were removed at various time points and separated on a 4-20% gradient gel followed by autoradiography. Alternately, aliquots were added to 10% trichloroacetic acid (TCA) or ddH20 on ice for 30 min and 2 mg/mL bovine serum albumin as a carrier. Samples were centrifuged at 12,000×g for 30 min, and the supernatant removed for analysis by scintillation spectrometry. The percent of degradation was determined by dividing the measured counts after TCA precipitation by the total counts measured in ddH20. To assess the ubiquitin-dependence of degradation, [35S]Sic1-Ubn was pretreated with 10 nM Usp2 cc (Baker et al., 2005) for 30 min at 37° C. prior to the addition of proteasomes. To inhibit substrate degradation, 26S proteasomes were pretreated with 10 μM MG132 (Biomol) prior to the addition of substrate. The initial rate of degradation was determined by least-squares linear regression analyses of 5, 10, and 20 min time points using Sigmaplot. The dependence of the initial rate of degradation on substrate concentration was plotted, and the data fit to the Michaelis-Menten equation using Sigmaplot.

Proteasome Inhibition Assay

The proteasome degradation inhibition assay was set up in 50 μL total volume containing 10 nM 26S proteasomes, 100 nM [35S]Sic1-Ubn, and 0-500 nM MPC-Ubn in 1×ATP buffer. 5 μL aliquots were removed at 5, 10 and 20 min for TCA precipitation and scintillation counting was carried out as described above. The initial rate of degradation as a percent of the control reaction is reported as the percent ratio of the initial rate of degradation of [35S]Sic1-Ubn in the presence of varying concentrations of MPC-Ubn and the initial rate of degradation for 35S]Sic1-Ubn in a control reaction with no MPC-Ubn. The dependence of the initial rate of degradation of [35S]Sic1-Ubn on the concentration of MPC-Ubn was plotted, and the data fit to the following equation for competitive inhibition using SigmaPlot: v0=(viK0.5)/(K0.5+[Ubn]), where K0.5=(1+[S]/KM)Ki, Ubn is the concentration of ubiquitinated inhibitor, and [S] is the concentration of [35S]Sic1-Ubn (Thrower et al., 2000).

In Vitro Formation of Ubiquitinated Huntingtin Aggregates

The TEV protease-induced cleavage and aggregation of GST-HttEx1-Q51-S and filter-trap analysis was carried out as previously described (Bennett et al., 2005). Aggregation of 0.15 mg/mL of ubiquitinated GST-PY-HttQ51-S was initiated by the addition of 1:1 (w/w) TEV protease and 1:1 (w/w) of GST-HttQ51ΔS in 1× aggregation buffer (40 mM Tris-HCl pH 7.2, 1 mM DTT, and 0.5 mM EDTA). The aggregation reaction was incubated at 37° C. and considered complete after 24 hrs. Filter trap analysis of ubiquitinated aggregates was carried out using both anti-ubiquitin antibody (clone FK2, Biomol) and an S-tag HRP conjugate followed by chemiluminescence. The degradation of 100 nM [35S]Sic1-Ubn in the presence of 0-625 nM ubiquitinated aggregates was assessed and the inhibition constant (Ki) determined as described above.

Microscopy

HEK293 cells were transfected with the indicated htt-chFP plasmid, and grown on poly-D-lysine coated coverslips. 72 hrs after transfection cells were fixed with 4% paraformaldehyde. Cells were imaged by epifluorescence on a Zeiss Axiovert 200M microscope with a ×100 oil lens (NA1.4; Zeiss). Digital (12-bit) images were acquired with a cooled CCD (Roper Scientific) and processed using Metamorph software (Universal Imaging). For live cell imaging, cells were grown in poly-D-lysine coated glass bottom dishes (MatTek Corporation, Ashland, Mass.) and were treated with 10 μM MG132. At the indicated time points images were acquired using a ×40 air objective (NA0.75; Zeiss) without prior fixation. Quantification of UPS reporter fluorescence in htt(Qn)-chFP expressing cells was performed as previously described (Bence et al., 2001; Bence et al., 2005).

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

### Proteostasis Collapse, not Proteasome Impairment, Leads to Accumulation of Polyubiquitin Chains in a Cellular Model of Huntington's Disease

This study investigated the mechanistic relationship between huntingtin aggregation and UPS impairment in cell culture and in vitro. Using a quantitative flow cytometry assay to simultaneously interrogate huntingtin expression and UPS function in single cells, we found that UPS reporters accumulate above a sharp and reproducible threshold of huntingtin expression that is inversely proportional to polyglutamine length. The observation that eight fluorescent 26S substrates bearing different Ub-dependent and Ub-independent degrons all accumulate above the same threshold level of huntingtin expression strongly suggests that huntingtin-induced UPS impairment occurs at the level of 26S, the only common factor to all substrates. However, in vitro studies using purified 26S indicate that, while polyubiquitinated huntingtin can directly inhibit proteasome activity in vitro by competing with other ubiquitinated substrates, the Ki with which huntingtin fragments inhibit purified 26S is independent of both polyglutamine length and aggregation state. Together, these findings lead us to conclude that protein aggregates do not choke or clog proteasomes and that persistent huntingtin aggregation leads to collapse of the cellular proteostasis network, leading to en masse diversion of proteins from productive folding pathways to become competitive substrates of 26S. This model is supported by our demonstration that both the extent of 26S impairment and the threshold of huntingtin concentration at which it occurs are strongly suppressed by environmental, pharmacological or genetic induction of heat shock factor 1 (HSF1), providing strong support for this transcriptional stress response pathway as a therapeutic target in HD and potentially other neurodegenerative diseases.

### Example 1

### UPS Substrates Accumulate Above a Critical Concentration Threshold of Mutant Huntingtin Expression

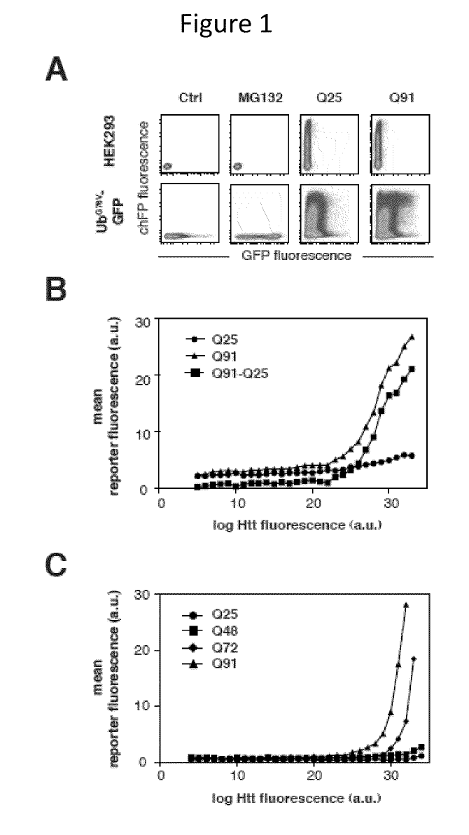

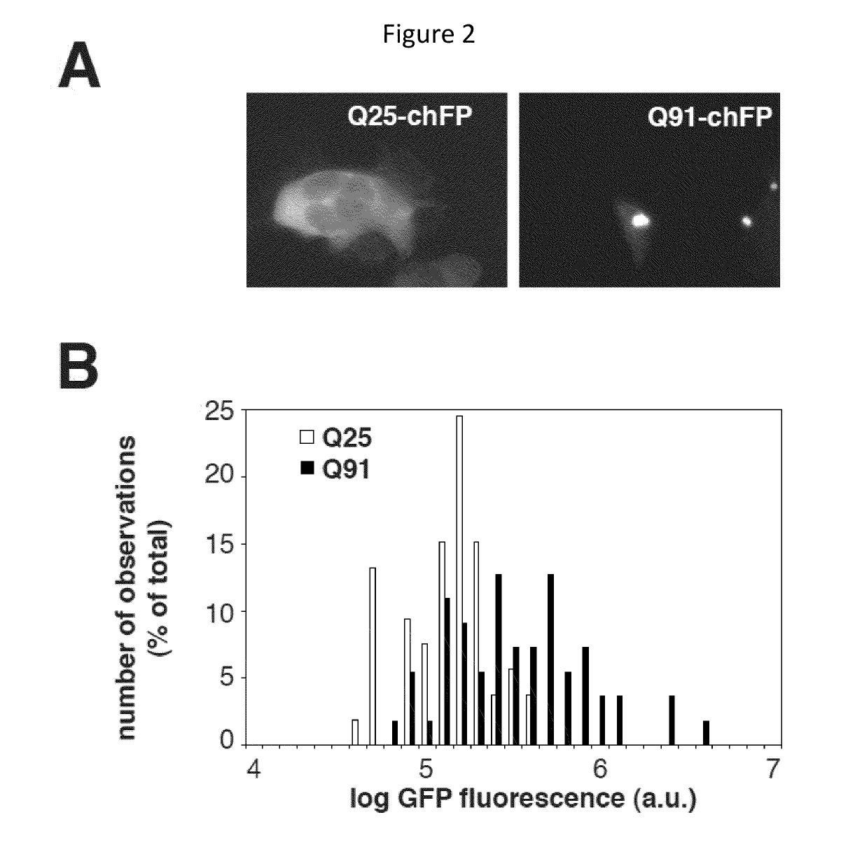

In order to assess a quantitative relationship between the amount of huntingtin expressed in cells and accumulation of a UPS reporter, a two-color flow cytometry-based assay was devised to simultaneously analyze UPS function using UbG76V, the mutant form of ubiquitin, coupled to the green fluorescent protein (GFP) as unstable green fluorescent UPS reporter, UbG76V-GFP (Dantuma et al., 2000), as well as huntingtin expression, using a red (“cherry”) fluorescent huntingtin exon 1 fragment, htt(Qn)-chFP (see FIG. 1). Control experiments established that the presence of chFP did not alter the aggregation properties of htt(Qn) (see FIG. 2, Panel A) or its impact on the UPS (see FIG. 2, Panel B). Because htt(Q25)-chFP or httQ(91)-chFP differ only in the length of the polyglutamine tract, the red fluorescence signal can be used to directly compare the expression levels of the two proteins (FIG. 1, Panel A, top). Expression of htt(Q25)-chFP in cells stably expressing UbG76V-GFP resulted in the appearance of a population of doubly-labeled cells in which the distribution of green fluorescence was not substantially different from that of untransfected reporter cells (see FIG. 1, Panel A, bottom). By contrast, expression of htt(Q91)-chFP resulted in the appearance of a distinct subpopulation of cells displaying high levels of both green and red fluorescence. Transformation of the data in FIG. 1, Panel A, to plot the concentration of htt(Qn)-chFP versus UbG76V-GFP fluorescence revealed that while htt(Q25)-chFP had only a minimal effect on the level of green fluorescence, htt(Q91)-chFP induced accumulation of the UPS reporter at high expression levels (see FIG. 1, Panel B). Details of the data transformation are presented in FIG. 3.

The htt(Q91)-specific effect, determined by subtracting green fluorescence in the presence of htt(Q25)-chFP from that of htt(Q91)-chFP, shows that UbG76V-GFP accumulates significantly only in cells expressing htt(Q91)-chFP above a sharp threshold (˜22 a.u.) (see FIG. 1, Panel B). This strikingly non-linear relationship between htt(Q91)-chFP expression and UPS impairment is reminiscent of the concentration-dependence of polyglutamine aggregation (or other polymerization reactions) in vitro, which exhibit exponential polymerization kinetics above their critical concentrations. To test this hypothesis, we examined the effect of polyglutamine length on the concentration-dependence of UPS reporter accumulation in response to htt(Qn) expression, as the critical concentration of polyglutamine peptide aggregation in vitro is strongly and non-linearly dependent on the number of glutamines (Chen et al., 2001). Indeed, the threshold concentration of htt(Qn)-GFP at which the UPS reporter, chFP-CL1, accumulates was inversely proportional to polyglutamine length (FIG. 1, Panel C). This glutamine length and concentration dependence suggests a strict relationship between huntingtin aggregation and UPS impairment that has been suggested in previous studies (Bence et al., 2001).

### Example 2

### Huntingtin Aggregation is Linked to Reduced Capacity of the 26S Proteasome

Stabilization of UbG76V-GFP by high levels of mutant huntingtin could be due to interference by aggregated or oligomeric protein with 26S, with some aspect of Ub conjugation, or both. To discriminate between these possibilities, we generated HEK293 cell lines stably expressing GFP molecules that are destabilized by degrons that target proteins to the proteasome by different Ub-dependent mechanisms. These reporters include, in addition to UbG76V-GFP (Dantuma et al., 2000), a substrate of the ubiquitin fusion degradation (UFD) pathway that recognizes proteins with an uncleavable N-terminal Ub (Johnson et al., 1995), Ub-Arg-GFP (Dantuma et al., 2000), a substrate of the N-end rule pathway that recognizes proteins with destabilizing N-terminal amino acids (Varshaysky, 1997), and GFP-CL1 (Bence et al., 2001), which is destabilized by a short amphipathic motif which, in yeast, targets proteins for ubiquitination by a distinct set of E2 and E3 enzymes (Gilon et al., 2000).

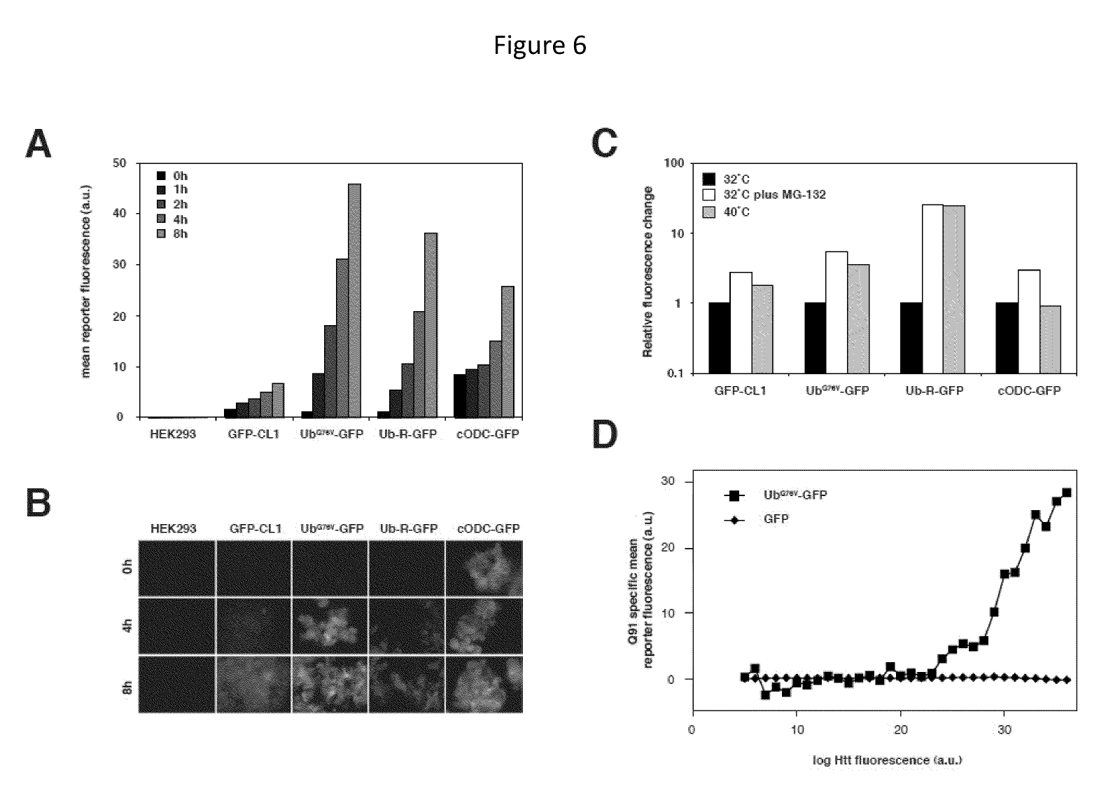

In addition, we examined the effect of htt(Qn)-chFP expression on the levels of cODC-GFP, a 26S substrate which is destabilized by an ubiquitin-independent degron consisting of the 37 C-terminal amino acids from ornithine decarboxylase (ODC) (Zhang et al., 2003). All four reporters, stably expressed in clonal HEK293 cell lines, exhibit low steady-state fluorescence in the absence of stress and diffuse time-dependent increases in cytoplasmic fluorescence following challenge with MG132 (see FIG. 6, Panels A and B). Control experiments using ts20 Balb/C 3T3 fibroblasts harboring a thermolabile E1 (Kulka et al., 1988) verified that the cODC-GFP fusion protein degradation is indeed subject to Ub-independent degradation by the proteasome (see FIG. 6, Panel C).

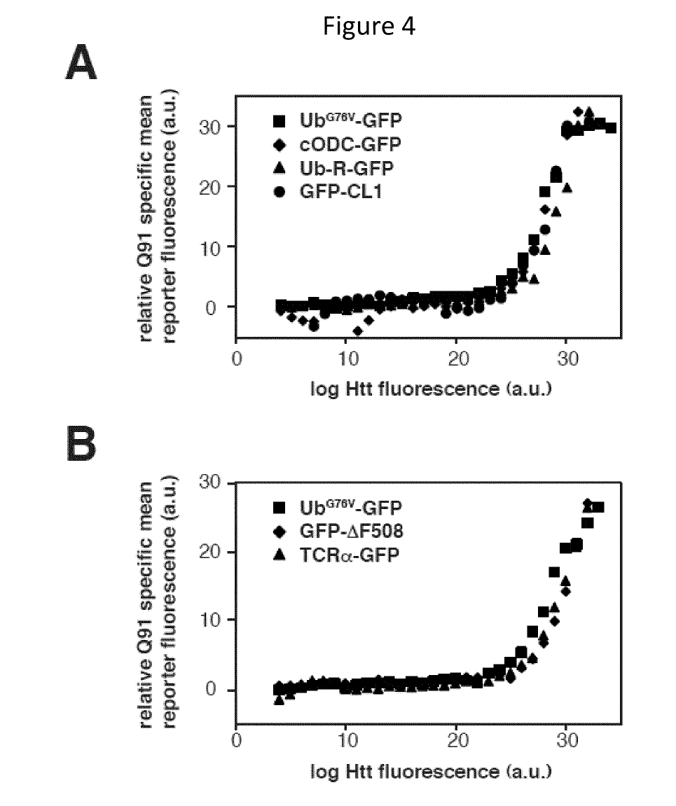

Expression of htt(Q91)-chFP, but not htt(Q25)-chFP, resulted in a htt(Q91)-chFP dose-dependent increase in the fluorescence of all four reporters (see FIG. 4, Panel A), strongly suggesting that expression of htt(Q91)-chFP impairs the UPS downstream of any Ub dependent targeting steps, mostly likely at the level of 26S itself. To further test this hypothesis, we assessed the effect of htt(Q91)-chFP expression on the degradation of substrates of endoplasmic reticulum-associated degradation (ERAD), a quality control pathway that delivers misfolded or unassembled proteins in the early secretory pathway to the UPS (Vembar and Brodsky, 2008). We found that htt(Q91)-chFP, but not htt(Q25)-chFP expression results in accumulation of folding defective mutant cystic fibrosis transmembrane conductance regulator (CFTR), GFP-ΔF508, (Ward et al., 1995) and of unassembled TCRα-GFP, a subunit of the T-cell receptor (Yu et al., 1997) (see FIG. 4, Panel B). Two additional ERAD substrates, the amyloidogenic mutant of the non-glycosylated protein transthyretin (TTR) (Sekijima et al., 2005) and GluR1, an unassembled subunit of the NMDA receptor, exhibited similar behavior (data not shown).

Remarkably, all of these ERAD substrates accumulate at precisely the same threshold of htt(Q91)-chFP expression as found for the four cytosolic UPS reporters (see FIG. 4, Panels A and B). It is possible that the elevated fluorescence of all of these unstable GFP conjugates could be due to increased transcription of the reporter cDNAs in response to htt(Q91)-chFP expression (Alvarez-Castelao et al., 2009; Bowman et al., 2005). However, we found no evidence of increased levels of GFP in response to the highest levels of htt(Q91)-chFP expression (see FIG. 6, Panel D). Because GFP is a very stable protein in mammalian cells (Bence et al., 2001), small changes in its rate of synthesis are integrated over time to generate robust changes in steady-state levels. The finding that a Ub-independent 26S substrate, cODC-GFP, along with substrates which depend on different Ub conjugation pathways accumulate in response to expression of htt(Q91chFP) above a critical concentration threshold suggests that UPS impairment by htt(Q91)-chFP aggregation is due to interference with the function of 26S and not to impaired Ub metabolism. The observation that all of the unstable reporters accumulate above the same level of htt(Q91)-chFP expression strongly suggests that mutant huntingtin expression above a critical concentration threshold has pleiotropic effects on the activity or capacity of 26S.

### Example 3

### Ubiquitinated Polyglutamine Aggregates do not Choke or Clog the 26S Proteasome

The most parsimonious model to explain why all UPS substrates accumulate in response to huntingtin aggregation is if aggregated huntingtin interacts directly with and inhibits 26S activity. Although we have previously shown that neither soluble nor aggregated polyglutamine peptides nor huntingtin fragments inhibit 20S or 26S proteasomes in vitro (Bennett et al., 2005), it is conceivable that if polyubiquitinated, these aggregates could antagonize 26S proteasomes by virtue of their affinity for Ub receptors but resistance to ATP-dependent unfolding associated with the 19S ATPases. In order to directly test the possibility that 26S proteasomes are inhibited in such a manner, we established an in vitro assay for 26S proteasome activity to measure the effect of polyubiquitination on the ability of aggregated polyglutamine-containing proteins to

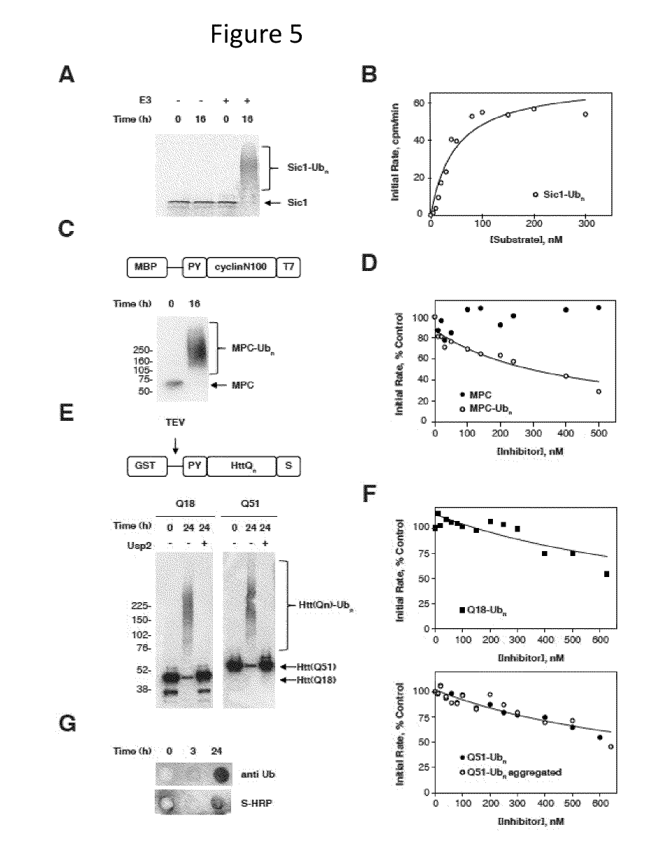

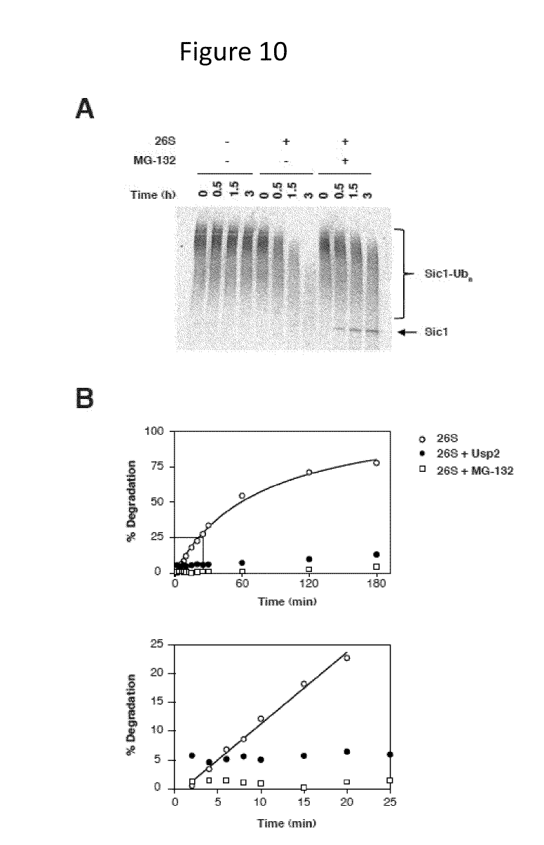

interfere with substrate degradation in trans. Purified [35S]-labeled substrate (PY-Sic1) was efficiently and quantitatively converted to a high molecular weight polyubiquitinated PY-Sic1(Ubn) form in vitro by the E3 Ub ligase Rsp5 (see FIG. 5, Panel A) (Saeki et al., 2005). The addition of purified proteasomes to this preparation resulted in efficient degradation that was dependent on ATP (data not shown) and inhibited by MG132 and by pretreatment of the PY-Sic1(Ubn) substrate with the deubiquitinating enzyme Usp2 (Catanzariti et al., 2004) (see FIG. 10, Panels A and B). Michaelis-Menten analysis of [35S]PY-Sic1(Ubn) degradation indicated a Km=46.9±3.5 nM (see FIG. 5, Panel B), in good agreement with previous studies with other 26S proteasome substrates in vitro (Thrower et al., 2000) and confirming that this model substrate can be used to quantify the activity of 26S in vitro.

To assess inhibition of 26S activity in vitro we used the same enzymatic strategy to generate high molecular weight polyubiquitinated forms of an unlabeled chimeric protein (MPC) consisting of an Nterminal maltose binding protein (MBP) fused to a fragment of Xenopus cyclin (cyclin N100) (Chen and Fang, 2001) and a T7 epitope tag (see FIG. 5, Panel C). The initial rate of [35S]PYSic1(Ubn) degradation by purified 26S rat liver proteasomes was strongly inhibited (Ki=150 nM) by MPC(Ubn) but not by non-ubiquitinated (see FIG. 5, Panel D) or deubiquitinated (not shown) MPC, confirming that ubiquitinated proteins can inhibit 26S function, and establishing the utility of this assay for the evaluation of proteasome inhibition by ubiquitinated huntingtin aggregates.

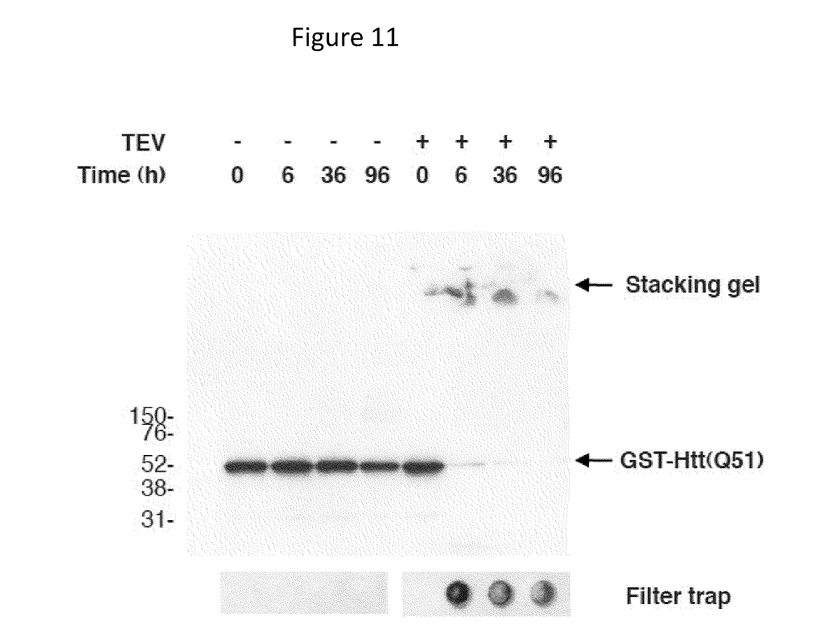

To assess the ability of ubiquitinated protein aggregates to inhibit 26S activity, we used a similar approach to append polyubiquitin chains on GST-PY-htt(Qn) purified from E. Coli. Previous studies have established that the presence of GST retards the aggregation of huntingtin fusions containing intermediate (˜Q50) length polyglutamine repeats, and that aggregation of Q>40 fusions can be efficiently induced upon cleavage of GST with a sitespecific protease (Scherzinger, 1997). Both GST-PY-htt(Q18) and GST-PY-htt(Q51) were efficiently converted to high molecular weight, Ub conjugated species. Treatment with Usp2 resolved both fusion proteins to their native, unmodified molecular weights indicating that their reduced mobility was due to Ub conjugation rather than to aggregation (see FIG. 5, Panel E). Hydrolysis of the GST moiety with TEV protease rapidly converted GST-PYhtt(Q51) into high molecular weight aggregates that did not migrate beyond the stacking gel on SDS-PAGE and were efficiently retained on a 0.2 μm cellulose acetate filter trap (Wanker et al., 1999) (see FIG. 11), confirming that the presence or absence of GST can be used to switch this fusion protein from a soluble non-aggregated protein into an aggregated state. We found that GST-htt(Q18) (Ubn) inhibited degradation of [35S]PY-Sic1(Ubn) by 26S in a dose-dependent manner (see FIG. 5, Panel F), with Ki ˜250 nM, similar to the value obtained for inhibition by MPC(Ubn).

Importantly, soluble GST-htt(Q51)(Ubn) and aggregated PY13 htt(Q51)(Ubn) (but not non-ubiquitinated forms (Bennett et al., 2005) and data not shown) also inhibited [35S]PY-Sic1(Ubn) with inhibitory constants 150-215 nM (see FIG. 5, Panel F), demonstrating that 26S inhibition is associated with the presence of a polyubiquitin chain on these proteins but is independent of their aggregation state. TEV-cleaved GSThtt(Q51)(Ubn)-S aggregates, retained on a 0.2 μm cellulose acetate filter, were immunoreactive with antibodies to Ub and S-protein, confirming the presence of polyubiquitinated huntingtin aggregates (see FIG. 5, Panel G). The similar Ki values for MPC(Ubn), GST-PY-htt(Q51)(Ubn) and aggregated PY-htt(Q51)(Ubn) support the conclusion that neither the presence of a polyglutamine tract nor the aggregation state influence the ability of a polyubiquitinated protein to inhibit the degradation of other ubiquitinated proteins by 26S in vitro. Therefore, although ubiquitinated huntingtin aggregates can compete with other ubiquitinated substrates for 26S, our data lead to the conclusion that, at least in vitro, proteasomes are neither choked nor clogged by huntingtin.

### Example 4

### Stabilization of Proteasome Reporters by Aggregation-Prone Huntingtin is not Due to Direct Competition for 26S

The in vivo data confirm an extremely strong dependence of 26S activity on polyglutamine expansion and concentration of co-expressed huntingtin, consistent with the conclusion that an aggregated form of huntingtin is the inhibitory species. However, inhibition of 26S proteasome activity by ubiquitinated proteins in vitro is independent of both the aggregation status and the length (or indeed the presence) of a polyglutamine tract. It is possible that ubiquitinated forms of huntingtin are present at sufficient concentrations in cells expressing polyglutamine expanded htt(Qn)-chFP to be able to outcompete other polyubiquitin-tagged substrates, including highly expressed GFP-reporters. However, if ubiquitinated huntingtin were to act as a simple competitor for binding of other ubiquitinated proteins to proteasomes, the concentration dependence of UbG76V-GFP stabilization should not exhibit such a steep threshold.

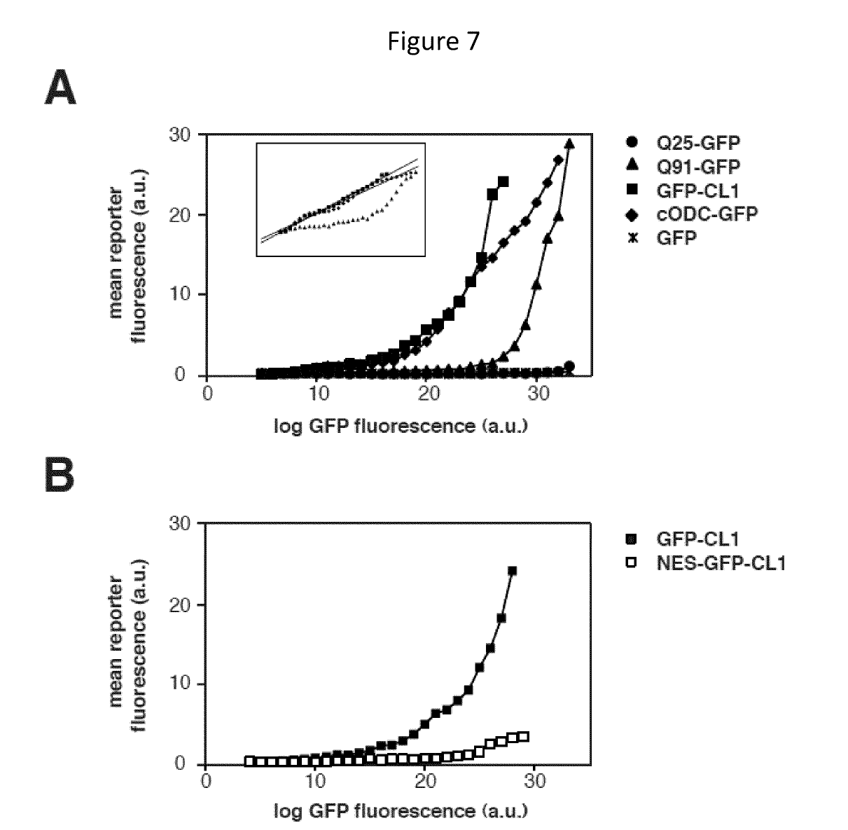

To test this, we examined the effect of expression of one short-lived, Ub-dependent fluorescent proteasome substrate on the accumulation of another (see FIG. 7, Panel A). We found that expression of GFP-CL1 or cODC-GFP resulted in accumulation of chFP-CL1 in a concentration-dependent manner but exhibited no sharp threshold, as reflected by the nearly linear relationship of the fluorescence profiles of the two proteins when represented on a double logarithmic plot (see FIG. 7, Panel A, inset). This suggests that two UPS substrates compete with each other for one or more essential limiting UPS component. Since the two CL1-tagged proteins share the same degron, they might compete with each other for limiting Ub conjugation machinery, but the observation that an Ub-independent proteasome substrate (cODC-GFP) interferes similarly with degradation of a Ub-dependent substrate (chFP-CL1) strongly suggests that the competition is at the level of proteasome binding, as the ODC degron functions as a molecular mimic which competes directly with Ub chains for binding to the 19S cap (Zhang et al., 2003). Therefore, competition for limited binding sites is a potential mechanism by which 26S activity might be limited by a highly expressed protein like htt(Qn)-chFP.

However, the lack of significant effect of htt(Q91)-chFP on 26S activity at concentrations well above those at which other short-lived UPS substrates are effective inhibitors (see FIG. 7, Panel A) indicates that either htt(Q91)-chFP must be somehow converted to become a potent direct 26S competitor above a precise polyglutamine- and concentration-dependent threshold, or that htt(Q91)-chFP inhibits 26S by a fundamentally different mechanism. For two proteins to compete, they must be present in the same cellular compartment as each other and with the activity for which they compete. chFP-CL1 is strongly stabilized by co-expression of GFP-CL1, which, like chFP-CL1, is expressed in both the nucleoplasm and cytoplasm (Bennett et al., 2005). By contrast, coexpression of NESGFP-CL1—which contains a potent nuclear export sequence and is strictly excluded from the nucleus (Bennett et al., 2005)—results in negligible accumulation chFP-CL1 (see FIG. 7, Panel B).

By contrast, expression of aggregation-prone proteins potently stabilizes nuclear or cytoplasmically-restricted GFP-CL1, irrespective of whether or not they are expressed in the same compartment (Bennett et al., 2005). These observations, together with the finding that huntingtin aggregates do not choke or clog proteasomes in vitro, lead us to propose an alternative, indirect model in which 26S becomes flooded with new substrates that are diverted to the UPS in the face of an overwhelming burden on the cellular proteostasis network imposed by the stress of maintaining huntingtin solubility.

### Example 4

### Failure of the Cellular Proteostasis Network to Maintain Huntingtin Solubility Leads to Accumulation of Ubiquitinated Proteasome Substrates

Proteostasis refers to the regulatory network that coordinates the expression of molecular chaperones, folding enzymes and degradative machinery with the amount and type of proteins present in a cell or cellular compartment under changing metabolic, environmental and developmentally programmed conditions (Balch et al., 2008). A key function of the proteostasis network is to ensure that expression of molecular chaperones is commensurate with the degree of “client” demand. To assess the dependence of proteasome impairment by htt(Qn)-chFP expression on proteostasis capacity, we used several paradigms to induce the cytoplasmic heat shock response, a transcriptional program which responds to cytoplasmic stress by activating the transcription of a large set of genes that includes most of the inducible cytoplasmic and nuclear chaperones.

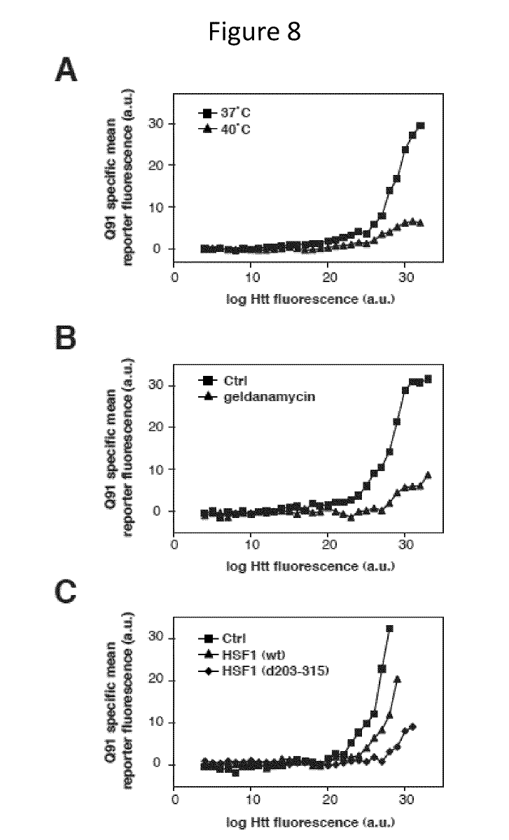

Subjecting cells to thermal stress (see FIG. 8, Panel A) or treatment with geldanamycin (see FIG. 8, Panel B), an HSP90 inhibitor and an indirect inducer of the heat shock response (Zou et al., 1998), shifted the threshold at which UbG76V-GFP accumulates to higher concentrations and attenuated the magnitude of the response of UPS reporter cells to htt(Q91)-chFP expression. Co-expression of the transcription factor HSF1, the master regulator of the mammalian heat shock response, resulted in a modest but reproducible shift of the htt(Q91)-chFP dose-response curve to the right (see FIG. 8, Panel C). However, expression of a constitutively active HSF1 variant, HSF1(d203-315) (Voellmy, 2005), exerted a dramatic effect on reporter accumulation similar to that observed with geldanamycin. Thus, activation of a gene or genes downstream of HSF1 can suppress the impairment of proteasome activity in response to expression of aggregation-prone huntingtin mutants.

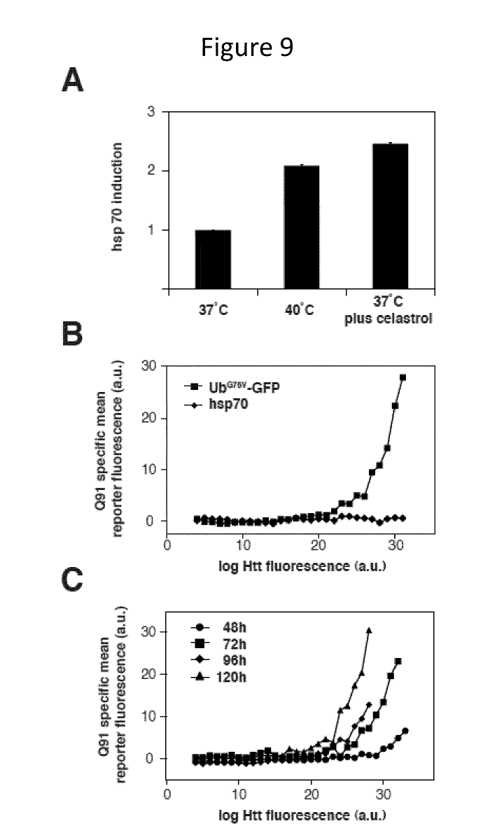

Given the effectiveness of HSF1 induction in suppressing UPS impairment caused by mutant huntingtin, we used a fluorescent reporter of HSF1 activity to assess the ability of cells to upregulate this cellular stress response pathway in response to htt(Q91)-chFP expression. We constructed a fluorescent reporter cell line consisting of GFP expressed under the control of the HSF1-repsonsive Hsp70 promoter (Hsp70::GFP). When HEK293 cells stably expressing this reporter were challenged with overnight exposure to elevated temperature or to treatment with celastrol, an established HSF1 inducer (Westerheide et al., 2004), mean GFP fluorescence increased (see FIG. 9, Panel A). Strikingly, expression of htt(Q91)-chFP in these cells did not result in any detectable increase in GFP fluorescence, even at the highest levels of htt(Q91)-chFP expression at which UbG76V-GFP levels were maximal (see FIG. 9, Panel B). Therefore, HEK293 cells are not able to respond to polyglutamine aggregation by upregulating the protective heat shock response. These data suggest that the stress of maintaining the solubility of this aggregation-prone protein leads to repression of the cellular heat shock response system. In support of this, we found that the same level of htt(Q91)-chFP expression had increasingly severe impact upon UPS function the longer the cells were forced to cope with htt(Q91)-chFP expression (see FIG. 9, Panel C). These data support the conclusion that UPS impairment is an indirect consequence of the collapse of cellular proteostasis in the face of chronic stress imposed by maintenance of mutant huntingtin solubility.

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Alvarez-Castelao, B., Martin-Guerrero, I., Garcia-Orad, A., and Castano, J. G. (2009). Cytomegalovirus promoter up-regulation is the major cause of increased protein levels of unstable reporter proteins after treatment of living cells with proteasome inhibitors. J Biol Chem 284, 28253-28262.
Baker, R. T., Catanzariti, A. M., Karunasekara, Y., Soboleva, T. A., Sharwood, R., Whitney, S., and Board, P. G. (2005). Using deubiquitylating enzymes as research tools. Methods Enzymol 398, 540-554.
Balch, W. E., Morimoto, R. I., Dillin, A., and Kelly, J. W. (2008). Adapting proteostasis for disease intervention. Science 319, 916-919.
Bence, N., Sampat, R., and Kopito, R. R. (2001). Impairment of the Ubiquitin-Proteasome System by Protein Aggregation. Science 292, 1552-1555.
Bence, N. F., Bennett, E. J., and Kopito, R. R. (2005). Application and Analysis of the GFP(u) Family of Ubiquitin-Proteasome System Reporters. Methods Enzymol 399, 481-490.
Bennett, E. J., Bence, N. F., Jayakumar, R., and Kopito, R. R. (2005). Global impairment of the ubiquitin-proteasome system by nuclear or cytoplasmic protein aggregates precedes inclusion body formation. Mol Cell 17, 351-365.
Bennett, E. J., Shaler, T. A., Woodman, B., Ryu, K. Y., Zaitseva, T. S., Becker, C. H., Bates, G. P., Schulman, H., and Kopito, R. R. (2007). Global changes to the ubiquitin system in Huntington's disease. Nature 448, 704-708.
Bett, J. S., Goellner, G. M., Woodman, B., Pratt, G., Rechsteiner, M., and Bates, G. P. (2006). Proteasome impairment does not contribute to pathogenesis in R6/2 Huntington's disease mice: exclusion of proteasome activator REGgamma as a therapeutic target. Hum Mol Genet 15, 33-44.
Bowman, A. B., Yoo, S. Y., Dantuma, N. P., and Zoghbi, H. Y. (2005). Neuronal dysfunction in a polyglutamine disease model occurs in the absence of ubiquitin-proteasome system impairment and inversely correlates with the degree of nuclear inclusion formation. Hum Mol Genet 14, 679-691.
Carrell R. W. & Lomas D. A. (1997). Conformational disease. Lancet 350 (9071), 134-138.
Catanzariti, A. M., Soboleva, T. A., Jans, D. A., Board, P. G., and Baker, R. T. (2004). An efficient system for high-level expression and easy purification of authentic recombinant proteins. Protein Sci 13, 1331-1339.
Chai, Y., Koppenhafer, S. L., Shoesmith, S. J., Perez, M. K., and Paulson, H. L. (1999). Evidence for proteasome involvement in polyglutamine disease: localization to nuclear inclusions in SCA3/MJD and suppression of polyglutamine aggregation in vitro. Hum Mol Genet 8, 673-682.
Chen, J., and Fang, G. (2001). MAD2B is an inhibitor of the anaphase-promoting complex. Genes Dev 15, 1765-1770.
Chen, S., Berthelier, V., Yang, W., and Wetzel, R. (2001). Polyglutamine aggregation behavior in vitro supports a recruitment mechanism of cytotoxicity. J Mol Biol 311, 173-182.
Cole, G. M., and Timiras, P. S. (1987). Ubiquitin-protein conjugates in Alzheimer's lesions. Neurosci Lett 79, 207-212.
Cummings, C. J., Mancini, M. A., Antalffy, B., DeFranco, D. B., Orr, H. T., and Zoghbi, H. Y. (1998). Chaperone suppression of aggregation and altered subcellular proteasome localization imply protein misfolding in SCA1. Nat Genet 19, 148-154.
Dantuma, N. P., Lindsten, K., Glas, R., Jellne, M., and Masucci, M. G. (2000). Short-lived green fluorescent proteins for quantifying ubiquitin/proteasome-dependent proteolysis in living cells. Nat Biotechnol 18, 538-543.
Deriziotis, P., and Tabrizi, S. J. (2008). Prions and the proteasome. Biochim Biophys Acta 1782, 713-722.
Diaz-Hernandez, M., Valera, A. G., Moran, M. A., Gomez-Ramos, P., Alvarez-Castelao, B., Castano, J. G., Hernandez, F., and Lucas, J. J. (2006). Inhibition of 26S proteasome activity by huntingtin filaments but not inclusion bodies isolated from mouse and human brain. J Neurochem 98, 1585-1596.
Fergusson, J., Landon, M., Lowe, J., Dawson, S. P., Layfield, R., Hanger, D. P., and Mayer, R. J. (1996). Pathological lesions of Alzheimer's disease and dementia with Lewy bodies brains exhibit immunoreactivity to an ATPase that is a regulatory subunit of the 26S proteasome. Neurosci Lett 219, 167-170.
Ferrer, I., Martin, B., Castano, J. G., Lucas, J. J., Moreno, D., and Olive, M. (2004). Proteasomal expression, induction of immunoproteasome subunits, and local MHC class I presentation in myofibrillar myopathy and inclusion body myositis. J Neuropathol Exp Neurol 63, 484-498.
Fujikake, N., Nagai, Y., Popiel, H. A., Okamoto, Y., Yamaguchi, M., and Toda, T. (2008). Heat shock transcription factor 1-activating compounds suppress polyglutamine-induced neurodegeneration through induction of multiple molecular chaperones. J Biol Chem 283, 26188-26197.
Fujimoto, M., Takaki, E., Hayashi, T., Kitaura, Y., Tanaka, Y., Inouye, S., and Nakai, A. (2005). Active HSF1 significantly suppresses polyglutamine aggregate formation in cellular and mouse models. J Biol Chem 280, 34908-34916.
Gidalevitz, T., Ben-Zvi, A., Ho, K. H., Brignull, H. R., and Morimoto, R. I. (2006). Progressive disruption of cellular protein folding in models of polyglutamine diseases. Science 311, 1471-1474.
Gidalevitz, T., Krupinski, T., Garcia, S., and Morimoto, R. I. (2009). Destabilizing protein polymorphisms in the genetic background direct phenotypic expression of mutant SOD1 toxicity. PLoS Genet 5, e1000399.
Gilon, T., Chomsky, O., and Kulka, R. G. (2000). Degradation signals recognized by the Ubc6p-Ubc7p ubiquitin-conjugating enzyme pair. Mol Cell Biol 20, 7214-7219. Hay, D. G., Sathasivam, K., Tobaben, S., Stahl, B., Marber, M., Mestril, R., Mahal, A., Smith, D. L.,
Woodman, B., and Bates, G. P. (2004). Progressive decrease in chaperone protein levels in a mouse model of Huntington's disease and induction of stress proteins as a therapeutic approach. Hum Mol Genet 13, 1389-1405.
Holmberg, C. I., Staniszewski, K. E., Mensah, K. N., Matouschek, A., and Morimoto, R. I. (2004). Inefficient degradation of truncated polyglutamine proteins by the proteasome. Embo J 23, 4307-4318.
Hsu, A. L., Murphy, C. T., and Kenyon, C. (2003). Regulation of aging and age-related disease by DAF-16 and heat-shock factor. Science 300, 1142-1145.
Illing, M. E., Rajan, R. S., Bence, N. F., and Kopito, R. R. (2002). A rhodopsin mutant linked to autosomal dominant retinitis pigmentosa is prone to aggregate and interacts with the ubiquitin proteasome system. J Biol Chem 277, 34150-34160.
Jana, N. R., Tanaka, M., Wang, G., and Nukina, N. (2000). Polyglutamine length-dependent interaction of Hsp40 and Hsp70 family chaperones with truncated N-terminal huntingtin: their role in suppression of aggregation and cellular toxicity. Hum Mol Genet 9, 2009-2018.
Johnson, E. S., Ma, P. C., Ota, I. M., and Varshaysky, A. (1995). A proteolytic pathway that recognizes ubiquitin as a degradation signal. J Biol Chem 270, 17442-17456.
Johnston, J. A., Ward, C. L., and Kopito, R. R. (1998). Aggresomes: a cellular response to misfolded proteins. J Cell Biol 143, 1883-1898.
Kawasaki, H., Murayama, S., Tomonaga, M., Izumiyama, N., and Shimada, H. (1987). Neurofibrillary tangles in human upper cervical ganglia. Morphological study with immunohistochemistry and electron microscopy. Acta Neuropathol 75, 156-159.
Kulka, R. G., Raboy, B., Schuster, R., Parag, H. A., Diamond, G., Ciechanover, A., and Marcus, M. (1988). A Chinese hamster cell cycle mutant arrested at G2 phase has a temperaturesensitive ubiquitin-activating enzyme, E1. J Biol Chem 263, 15726-15731.
Lehman, N. L. (2009). The ubiquitin proteasome system in neuropathology. Acta Neuropathol 118, 329-347.
Leverenz, J. B., Umar, I., Wang, Q., Montine, T. J., McMillan, P. J., Tsuang, D. W., Jin, J., Pan, C., Shin, J., Zhu, D., et al. (2007). Proteomic identification of novel proteins in cortical lewy bodies. Brain Pathol 17, 139-145.
Mandrusiak, L. M., Beitel, L. K., Wang, X., Scanlon, T. C., Chevalier-Larsen, E., Merry, D. E., and Trifiro, M. A. (2003). Transglutaminase potentiates ligand-dependent proteasome dysfunction induced by polyglutamine-expanded androgen receptor. Hum Mol Genet 12, 1497-1506.
Mayer, R. J., Lowe, J., Lennox, G., Doherty, F., and Landon, M. (1989). Intermediate filaments and ubiquitin: a new thread in the understanding of chronic neurodegenerative diseases. Prog Clin Biol Res 317, 809-818.
Mori, H., Kondo, J., and Ihara, Y. (1987). Ubiquitin is a component of paired helical filaments in Alzheimer's disease. Science 235, 1641-1644.
Morley, J. F., and Morimoto, R. I. (2004). Regulation of longevity in Caenorhabditis elegans by heat shock factor and molecular chaperones. Mol Biol Cell 15, 657-664.
Muchowski, P. J., Schaffar, G., Sittler, A., Wanker, E. E., Hayer-Hartl, M. K., and Hartl, F. U. (2000). Hsp70 and Hsp40 chaperones can inhibit self-assembly of polyglutamine proteins into amyloid-like fibrils. Proc Natl Acad Sci USA 97, 7841-7846.
Perry, G., Friedman, R., Shaw, G., and Chau, V. (1987). Ubiquitin is detected in neurofibrillary tangles and senile plaque neurites of Alzheimer disease brains. Proc Natl Acad Sci USA 84, 3033-3036.
Persichetti, F., Carlee, L., Faber, P. W., McNeil, S. M., Ambrose, C. M., Srinidhi, J., Anderson, M., Barnes, G. T., Gusella, J. F., and MacDonald, M. E. (1996). Differential expression of normal and mutant Huntington's disease gene alleles. Neurobiol Dis 3, 183-190.
Petrucelli, L., O'Farrell, C., Lockhart, P. J., Baptista, M., Kehoe, K., Vink, L., Choi, P., Wolozin, B., Farrer, M., Hardy, J., et al. (2002). Parkin protects against the toxicity associated with mutant alpha-synuclein: proteasome dysfunction selectively affects catecholaminergic neurons. Neuron 36, 1007-1019.
Pratt, G., and Rechsteiner, M. (2008). Proteasomes cleave at multiple sites within polyglutamine tracts: activation by PA28gamma(K188E).
J Biol Chem 283, 12919-12925.
Ryu, K. Y., Baker, R. T., and Kopito, R. R. (2006). Ubiquitin-specific protease 2 as a tool for quantification of total ubiquitin levels in biological specimens.
Anal Biochem 353, 153-155.
Ryu, K. Y., Garza, J. C., Lu, X. Y., Barsh, G. S., and Kopito, R. R. (2008). Hypothalamic neurodegeneration and adult-onset obesity in mice lacking the Ubb polyubiquitin gene. Proc Natl Acad Sci USA 105, 4016-4021.
Saeki, Y., Isono, E., and Toh, E. A. (2005). Preparation of ubiquitinated substrates by the PY motif-insertion method for monitoring 26S proteasome activity.
Methods Enzymol 399, 215-227.
Saigoh, K., Wang, Y. L., Suh, J. G., Yamanishi, T., Sakai, Y., Kiyosawa, H., Harada, T., Ichihara, N., Wakana, S., Kikuchi, T., et al. (1999). Intragenic deletion in the gene encoding ubiquitin carboxy-terminal hydrolase in gad mice. Nat Genet 23, 47-51.
Scherzinger, E., Lurz, R., Turmaine, M., Mangiarini, L., Hollenbach, B., Hasenbank, R., Bates, G., Davies, S. W., Lehrach, H. and E. Wanker (1997). Huntingtin-Encoded Polyglutamine Expansions Form Amyloid-like Protein Aggregates in Vitro and in Vivo. Cell 90, 549-558.
Sekijima, Y., Wiseman, R. L., Matteson, J., Hammarstrom, P., Miller, S. R., Sawkar, A. R., Balch, W. E., and Kelly, J. W. (2005). The biological and chemical basis for tissue-selective amyloid disease. Cell 121, 73-85.
Sittler, A., Lurz, R., Lueder, G., Priller, J., Lehrach, H., Hayer-Hartl, M. K., Hartl, F. U., and Wanker, E. E. (2001). Geldanamycin activates a heat shock response and inhibits huntingtin aggregation in a cell culture model of Huntington's disease.
Hum Mol Genet 10, 1307-1315.
Steffan, J. S., Agrawal, N., Pallos, J., Rockabrand, E., Trotman, L. C., Slepko, N., Illes, K., Lukacsovich, T., Zhu, Y. Z., Cattaneo, E., et al. (2004). SUMO modification of Huntingtin and Huntington's disease pathology. Science 304, 100-104.
Stenoien, D. L., Cummings, C. J., Adams, H. P., Mancini, M. G., Patel, K., DeMartino, G. N., Marcelli, M., Weigel, N. L., and Mancini, M. A. (1999). Polyglutamine-expanded androgen receptors form aggregates that sequester heat shock proteins, proteasome components and SRC-1, and are suppressed by the HDJ-2 chaperone.
Hum Mol Genet 8, 731-741.
Suhr, S. T., Senut, M. C., Whitelegge, J. P., Faull, K. F., Cuizon, D. B., and Gage, F. H. (2001). Identities of sequestered proteins in aggregates from cells with induced polyglutamine expression. J Cell Biol 153, 283-294.
Thrower, J. S., Hoffman, L., Rechsteiner, M., and Pickart, C. M. (2000). Recognition of the polyubiquitin proteolytic signal. Embo J 19, 94-102.
Trinklein, N. D., Murray, J. I., Hartman, S. J., Botstein, D., and Myers, R. M. (2004). The role of heat shock transcription factor 1 in the genome-wide regulation of the mammalian heat shock response. Mol Biol Cell 15, 1254-1261.
van Tijn, P., de Vrij, F. M., Schuurman, K. G., Dantuma, N. P., Fischer, D. F., van Leeuwen, F. W., and Hol, E. M. (2007). Dose-dependent inhibition of proteasome activity by a mutant ubiquitin associated with neurodegenerative disease. J Cell Sci 120, 1615-1623.
Varshaysky, A. (1997). The N-end rule pathway of protein degradation.
Genes Cells 2, 13-28.
Vembar, S. S., and Brodsky, J. L. (2008). One step at a time: endoplasmic reticulumassociated degradation. Nat Rev Mol Cell Biol 9, 944-957.
Venkatraman, P., Wetzel, R., Tanaka, M., Nukina, N., and Goldberg, A. L. (2004). Eukaryotic proteasomes cannot digest polyglutamine sequences and release them during degradation of polyglutamine-containing proteins. Mol Cell 14, 95-104.
Voellmy, R. (2004). On mechanisms that control heat shock transcription factor activity in metazoan cells. Cell Stress Chaperones 9, 122-133.
Voellmy, R. (2005). Dominant-positive and dominant-negative heat shock factors. Methods 35, 199-207.
Wang, J., Gines, S., MacDonald, M. E., and Gusella, J. F. (2005). Reversal of a full-length mutant huntingtin neuronal cell phenotype by chemical inhibitors of polyglutaminemediated aggregation. BMC Neurosci 6, 1.
Wanker, E. E., Scherzinger, E., Heiser, V., Sittler, A., Eickhoff, H., and Lehrach, H. (1999). Membrane filter assay for detection of amyloid-like polyglutamine-containing protein aggregates. Methods Enzymol 309, 375-386.
Ward, C. L., Omura, S., and Kopito, R. R. (1995). Degradation of CFTR by the ubiquitin proteasome pathway. Cell 83, 121-127.
Warrick, J. M., Chan, H. Y., Gray-Board, G. L., Chai, Y., Paulson, H. L., and Bonini, N. M. (1999). Suppression of polyglutamine-mediated neurodegeneration in Drosophila by the molecular chaperone HSP70. Nat Genet 23, 425-428.
Westerheide, S. D., Anckar, J., Stevens, S. M., Jr., Sistonen, L., and Morimoto, R. I. (2009). Stress-inducible regulation of heat shock factor 1 by the deacetylase SIRT1.
Science 323, 1063-1066.
Westerheide, S. D., Bosman, J. D., Mbadugha, B. N., Kawahara, T. L., Matsumoto, G., Kim, S., Gu, W., Devlin, J. P., Silverman, R. B., and Morimoto, R. I. (2004). Celastrols as inducers of the heat shock response and cytoprotection. J Biol Chem 279, 56053-56060.
Wigley, W. C., Fabunmi, R. P., Lee, M. G., Marino, C. R., Muallem, S., DeMartino, G. N., and Thomas, P. J. (1999). Dynamic association of proteasomal machinery with the centrosome. J Cell Biol 145, 481-490.
Wilson, S. M., Bhattacharyya, B., Rachel, R. A., Coppola, V., Tessarollo, L., Householder, D. B., Fletcher, C. F., Miller, R. J., Copeland, N. G., and Jenkins, N. A. (2002). Synaptic defects in ataxia mice result from a mutation in Usp14, encoding a ubiquitin-specific protease. Nat Genet 32, 420-425.
Woodman, B., Butler, R., Landles, C., Lupton, M. K., Tse, J., Hockly, E., Moffitt, H., Sathasivam, K., and Bates, G. P. (2007). The Hdh(Q150/Q150) knock-in mouse model of HD and the R6/2 exon 1 model develop comparable and widespread molecular phenotypes.
Brain Res Bull 72, 83-97.
Xia, Q., Liao, L., Cheng, D., Duong, D. M., Gearing, M., Lah, J. J., Levey, A. I., and Peng, J. (2008). Proteomic identification of novel proteins associated with Lewy bodies.
Front Biosci 13, 3850-3856.
Yu, H., Kaung, G., Kobayashi, S., and Kopito, R. R. (1997). Cytosolic degradation of T-cell receptor alpha chains by the proteasome. J Biol Chem 272, 20800-20804.
Zhang, M., Pickart, C. M., and Coffino, P. (2003). Determinants of proteasome recognition of ornithine decarboxylase, a ubiquitin-independent substrate. EMBO J 22, 1488-1496.
Zhang, Y. Q., and Sarge, K. D. (2007). Celastrol inhibits polyglutamine aggregation and toxicity though induction of the heat shock response. J Mol Med 85, 1421-1428.
Zou, J., Guo, Y., Guettouche, T., Smith, D. F., and Voellmy, R. (1998). Repression of heat shock transcription factor HSF1 activation by HSP90 (HSP90 complex) that forms a stress sensitive complex with HSF1. Cell 94, 471-480.

What is claimed is:

1. A method of identifying a regulator of cellular protein homeostasis, the method comprising:
providing a cell expressing a ubiquitin-proteasome system (UPS) reporter and an aggregation-prone protein;
contacting said cell with a candidate agent; and
analyzing said UPS reporter expression in presence of said candidate agent;
comparing said UPS reporter expression in presence of said candidate agent with UPS reporter expression in absence of said candidate expression; and
assessing said candidate agent's potential as regulator of cellular protein homeostasis.

2. The method of claim 1, wherein the aggregation-prone protein is huntingtin.

3. The method of claim 1, wherein said UPS reporter carries a fluorescent label.

4. The method of claim 1, wherein said UPS reporter carries a label for nonfluorescent detection.

5. The method of claim 1, wherein the cell is mammalian.

6. A method of reducing aggregation of an aggregation-prone protein, the method comprising administering to a mammal a regulator of cellular protein homeostasis identified in accordance to claim 1.

7. The method of claim 6, wherein the aggregation-prone protein is huntingtin.

8. The method of claim 6, wherein the regulator of cellular protein homeostasis is a small interfering RNA (siRNA).

9. The method of claim 6, wherein the regulator of cellular protein homeostasis is a complementary deoxyribonucleic acid (cDNA).

10. The method of claim 6, wherein the regulator of cellular protein homeostasis is a small molecule.
